(19) United States
(12) Patent Application Publication
(10) Pub. No.: US 2026/0258015 A1
(43) Pub. Date: Sep. 3, 2026

(54) CRYSTALLINE FORMS OF 2-(4-(4-(AMINOMETHYL)-1-OXO-1,2- DIHYDROPHTHALAZIN-6-YL)-1-METHYL-1H- PYRAZOL-5-YL)-4-CHLORO-6-CYCLOPROPOXY-3-FLUOROBENZONITRILE

(71) Applicant: MIRATI THERAPEUTICS, INC., PRINCETON, NJ (US)

(72) Inventors: MICHAL ACHMATOWICZ, SAN DIEGO, CA (US); SVITLANA KULYK, SAN DIEGO, CA (US); CHRISTOPHER RONALD SMITH, SAN DIEGO, CA (US); NAN WANG, SOUTH PLAINFIELD, NJ (US); HARSH SHAH, SOUTH PLAINFIELD, NJ (US); TIAN XIE, SOUTH PLAINFIELD, NJ (US); QI GAO, SOUTH PLAINFIELD, NJ (US); NIKHIL LOKA, ESCONDIDO, CA (US)

(73) Assignee: MIRATI THERAPEUTICS, INC., PRINCETON, NJ (US)

(21) Appl. No.: 19/155,574

(22) PCT Filed: Feb. 12, 2024
(86) PCT No.: PCT/US2024/015345

§ 371 Date: Aug. 12, 2025

Related U.S. Application Data
(60) Provisional application No. 63/484,604, filed on Feb. 13, 2023.

(51) Int. Cl. C07D 403/10

(52) U.S. Cl. CPC C07D 403/10

(57) ABSTRACT

The present invention relates to crystalline forms of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile salt polymorphs, pharmaceutically acceptable compositions comprising these crystalline forms, and methods for using these crystalline forms.

### CROSS-REFERENCE TO RELATED APPLICATIONS

This application claims priority from U.S. Provisional Application No. 63/484,604, filed Feb. 13, 2023, the disclosure of which is hereby incorporated by reference in its entirety.

### FIELD OF THE INVENTION

This invention relates to salts of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile and particular crystalline forms thereof, processes of preparing the crystalline forms, and pharmaceutical compositions including the crystalline forms. The crystalline forms thereof are useful in the treatment and/or prevention of diseases and/or conditions related to cell proliferation, such as cancer. In particular, the crystalline forms provide therapeutic benefits as MTA-cooperative inhibitors of Protein Arginine N-Methyl Transferase 5 (PRMT5).

### BACKGROUND OF THE INVENTION

Protein Arginine N-Methyl Transferase (PRMT5) is a Form II arginine methyltransferase that catalyzes the transfer of a methyl group from S-adenosyl-L-methionine (SAM) to an omega-nitrogen of the guanidino function of protein L-arginine residues (omega-monomethylation) and the transfer of a second methyl group to the other omega-nitrogen, yielding symmetric dimethylarginine (sDMA). PRMT5 forms a complex with MEP50 (methylosome protein 50), which is required for substrate recognition and orientation and is also required for PRMT5-catalyzed histone 2A and histone 4 methyltransferase activity (e.g., see Ho et al., (2013) PLOS ONE 8 (8): 10.1371/annotation/e6b5348e-9052-44ab-8f06-90d01dc88fc2).

Homozygous deletions of p16/CDKN2a are prevalent in cancer and these mutations commonly involve the co-deletion of adjacent genes, including the gene encoding methylthioadenosine phosphorylase (MTAP). It is estimated that approximately 15% of all human cancers have a homozygous deletion of the MTAP gene (e.g., see Firestone & Schramm (2017) J. Am. Chem Soc. 139 (39): 13754-13760. doi: 10.1021/jacs.7b05803. Epub 2017 Sep. 20).

Cells lacking MTAP activity have elevated levels of the MTAP substrate, methylthioadenosine (MTA), which is a potent inhibitor of PRMT5. Inhibition of PRMT5 activity results in reduced methylation activity and increased sensitivity of cellular proliferation to PRMT5 depletion or loss of activity. Hence, the loss of MTAP activity reduces methylation activity of PRMT5 making the cells selectively dependent on PRMT5 activity.

Thus, MTA-cooperative inhibition of PRMT5 activity in MTAP deleted cancers can provide therapeutic benefit for a wide range of cancers. The compounds of the invention provide this therapeutic benefit as MTA-cooperative inhibitors of PRMT5 that negatively modulate the activity of MTA-bound PRMT5 in a cell, particularly an MTAP-deficient cell, or for treating various forms of MTAP-associated cancer.

In particular, 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile, is a potent and selective inhibitors of PRMT5 and has been found to be pharmacologically active. Here, crystalline salts, including sulfates, tosylate, glutamates, fumarates, glycolates, hippurates, and phosphates of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile have been found that are suitable for use in pharmaceutical compositions.

### SUMMARY OF THE INVENTION

In one aspect, this disclosure provide crystalline forms of salts of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile, shown below, and hereinafter Compound 1.

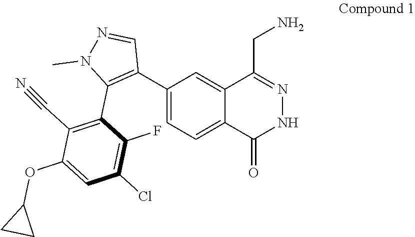

The salts of compound 1 may be selected from sulfate, tosylate, glutamate, fumarate, glycolate, hippurate, and phosphate.

In another aspect, this disclosure also provides a particular crystalline form of Compound 1, hereinafter sulfate salt Form A. This disclosure further provides processes of preparing sulfate salt Form A. This disclosure further provides pharmaceutical compositions comprising sulfate salt Form A and a pharmaceutically acceptable carrier.

In another aspect, this disclosure also provides a particular crystalline form of Compound 1, hereinafter tosylate salt Form A. This disclosure further provides processes of preparing tosylate salt Form A. This disclosure further provides pharmaceutical compositions comprising tosylate salt Form A and a pharmaceutically acceptable carrier.

In another aspect, this disclosure also provides a particular crystalline form of Compound 1, hereinafter glutamate salt Form A. This disclosure further provides processes of preparing glutamate salt Form A. This disclosure further provides pharmaceutical compositions comprising glutamate salt Form A and a pharmaceutically acceptable carrier.

In another aspect, this disclosure also provides a particular crystalline form of Compound 1, hereinafter fumarate salt Form A. This disclosure further provides processes of preparing fumarate salt Form A. This disclosure further provides pharmaceutical compositions comprising fumarate salt Form A and a pharmaceutically acceptable carrier.

In another aspect, this disclosure also provides a particular crystalline form of Compound 1, hereinafter fumarate salt Form B. This disclosure further provides processes of preparing fumarate salt Form B. This disclosure further provides pharmaceutical compositions comprising fumarate salt Form B and a pharmaceutically acceptable carrier.

In another aspect, this disclosure also provides a particular crystalline form of Compound 1, hereinafter glycolate salt Form A. This disclosure further provides processes of preparing glycolate salt Form A. This disclosure further provides pharmaceutical compositions comprising glycolate salt Form A and a pharmaceutically acceptable carrier.

In another aspect, this disclosure also provides a particular crystalline form of Compound 1, hereinafter glycolate salt Form B. This disclosure further provides processes of preparing glycolate salt Form B. This disclosure further provides pharmaceutical compositions comprising glycolate salt Form B and a pharmaceutically acceptable carrier.

In another aspect, this disclosure also provides a particular crystalline form of Compound 1, hereinafter hippurate salt Form A. This disclosure further provides processes of preparing hippurate salt Form A. This disclosure further provides pharmaceutical compositions comprising hippurate salt Form A and a pharmaceutically acceptable carrier.

In another aspect, this disclosure also provides a particular crystalline form of Compound 1, hereinafter phosphate salt Form A. This disclosure further provides processes of preparing phosphate salt Form A. This disclosure further provides pharmaceutical compositions comprising phosphate salt Form A and a pharmaceutically acceptable carrier.

This disclosure further provides methods of treating cancer comprising administering to a subject in need of such treatment a crystalline form of Compound 1 as disclosed herein or a pharmaceutical composition comprising a crystalline form of Compound 1.

### BRIEF DESCRIPTION OF THE FIGURES

FIG. 1A is an X-ray powder diffraction (XRPD) pattern of sulfate salt Form A solid of Compound 1.

FIG. 2A is a differential scanning calorimetric (DSC) thermogram and a thermal gravimetric analysis (TGA) overlay of tosylate salt Form A solid of Compound 1.

FIG. 2B is an X-ray powder diffraction (XRPD) pattern of tosylate salt Form A solid of Compound 1.

FIG. 3A is a differential scanning calorimetric (DSC) thermogram and a thermal gravimetric analysis (TGA) overlay of glutamate salt Form A solid of Compound 1.

FIG. 3B is an X-ray powder diffraction (XRPD) pattern of glutamate salt Form A solid of Compound 1.

FIG. 4A is a differential scanning calorimetric (DSC) thermogram and a thermal gravimetric analysis (TGA) overlay of fumarate salt Form A solid of Compound 1.

FIG. 4B is X-ray powder diffraction (XRPD) patterns of fumarate salt Form A solid of Compound 1.

FIG. 4C is a differential scanning calorimetric (DSC) thermogram and a thermal gravimetric analysis (TGA) overlay of fumarate salt Form B solid of Compound 1.

FIG. 4D is X-ray powder diffraction (XRPD) patterns of fumarate salt Form B solid of Compound 1.

FIG. 5A is a differential scanning calorimetric (DSC) thermogram and a thermal gravimetric analysis (TGA) overlay of glycolate Form A solid of Compound 1.

FIG. 5B is X-ray powder diffraction (XRPD) patterns of glycolate salt Form A solid of Compound 1.

FIG. 5C is a differential scanning calorimetric (DSC) thermogram and a thermal gravimetric analysis (TGA) overlay of glycolate salt Form B solid of Compound 1.

FIG. 5D is X-ray powder diffraction (XRPD) patterns of glycolate salt Form B solid of Compound 1.

FIG. 6A is a differential scanning calorimetric (DSC) thermogram and a thermal gravimetric analysis (TGA) overlay of hippurate salt Form A solid of Compound 1.

FIG. 6B is a X-ray powder diffraction (XRPD) pattern of hippurate salt Form A solid of Compound 1.

FIG. 6C is a polarized light microscopy (PLM) image of hippurate salt Form A solid of Compound 1.

FIG. 7A is a differential scanning calorimetric (DSC) thermogram and a thermal gravimetric analysis (TGA) overlay of phosphate salt Form A solid of Compound 1.

FIG. 7B is a X-ray powder diffraction (XRPD) pattern of phosphate Form A solid of Compound 1.

FIG. 8A is an X-ray powder diffraction (XRPD) pattern of an amorphous Free Base solid of Compound 1.

FIG. 8B is a polarized light microscopy (PLM) image of an amorphous Free Base solid of Compound 1.

FIG. 8C is a differential scanning calorimetric (DSC) thermogram and a thermal gravimetric analysis (TGA) overlay of an amorphous Free Base solid of Compound 1.

FIG. 8D is a proton nuclear magnetic resonance (1H-NMR) spectrum of an amorphous Free Base solid of Compound 1.

### DETAILED DESCRIPTION OF THE INVENTION

As noted above, the invention provides particular crystalline forms of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile salts. In various embodiments, the salts may be selected from sulfate, tosylate, glutamate, fumarate, glycolate, hippurate, and phosphate.

In some embodiments, the invention also provides sulfate salt Form A, i.e., a particular crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile sulfate.

In some embodiments, the invention also provides tosylate salt Form A, i.e., a particular crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile tosylate.

In some embodiments, the invention also provides glutamate salt Form A, i.e., a particular crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glutamate.

In some embodiments, the invention also provides fumarate salt Form A, i.e., a particular crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile fumarate.

In some embodiments, the invention also provides fumarate salt Form B i.e., a particular crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile fumarate.

In some embodiments, the invention also provides glycolate salt Form A, i.e., a particular crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glycolate.

In some embodiments, the invention also provides glycolate salt Form B, i.e., a particular crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glycolate.

In some embodiments, the invention also provides hippurate salt Form A, i.e., a particular crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hippurate.

In some embodiments, the invention also provides phosphate salt Form A, i.e., a particular crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile phosphate.

The crystalline forms as described herein may be characterized using a number of methods known to the person of ordinary skill in the art including thermal analysis (e.g., differential scanning calorimetry (DSC), thermal gravimetric analysis (TGA)), X-ray powder diffraction (XRPD), microscopy (e.g., scanning electron microscopy (SEM), polarized light microscopy (PLM)), and spectroscopy (e.g., infrared, Raman, solid-sate nuclear magnetic resonance, proton nuclear magnetic resonance (1H-NMR)). The particle size and size distribution may be determined by conventional methods, such as laser light scattering technique. The purity of the crystalline forms provided herein may be determined by standard analytical methods, such as thin layer chromatography (TLC), gel electrophoresis, gas chromatography, high performance liquid chromatography (HPLC), and mass spectroscopy (MS).

### Sulfate Salt Form A

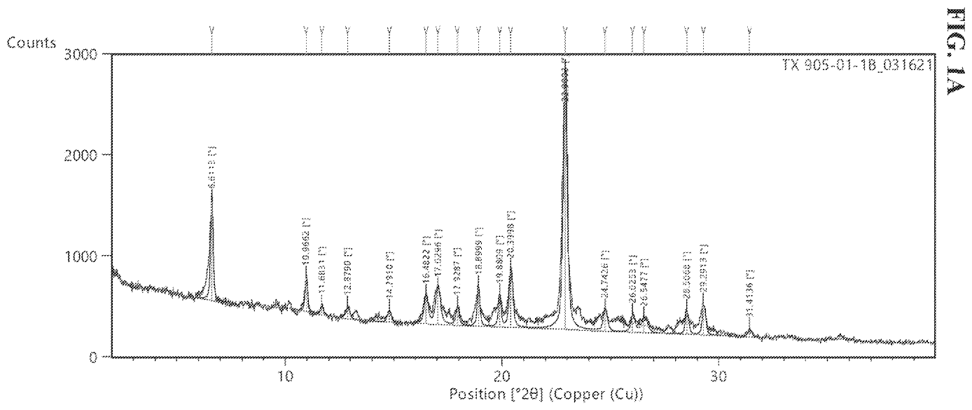

In one embodiment this disclosure provides a crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile sulfate, i.e., sulfate salt Form A. In various embodiments, sulfate salt Form A has an X-ray powder diffraction (XRPD) pattern. In some embodiments, sulfate salt Form A has an XRPD pattern comprising a peak at a two-theta angle of 6.6°±0.2° In some embodiments, sulfate salt Form A has an XRPD pattern comprising a peak at a two-theta angle of 10.9°±0.2°. In some embodiments, sulfate salt Form A has an XRPD pattern comprising a peak at a two-theta angle of 20.4°±0.2°. In some embodiments, sulfate salt Form A has an XRPD pattern comprising a peak at a two-theta angle of 22.9°±0.2°. In some embodiments, sulfate salt Form A has an XRPD pattern comprising peaks at two-theta angles of 6.6°±0.2°, 10.9°±0.2°, 20.4°±0.2°, and 22.9°±0.2°. For example, in some embodiments, sulfate salt Form A has an XRPD pattern substantially shown in FIG. 1A.

In some embodiments as described herein, sulfate salt Form A has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile sulfate. In some embodiments, sulfate salt Form A has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile sulfate.

### Tosylate Salt Form A

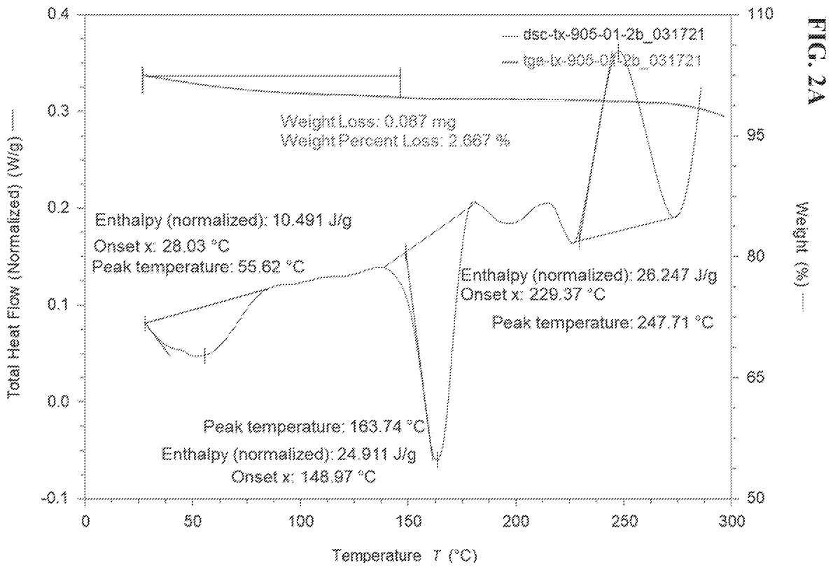

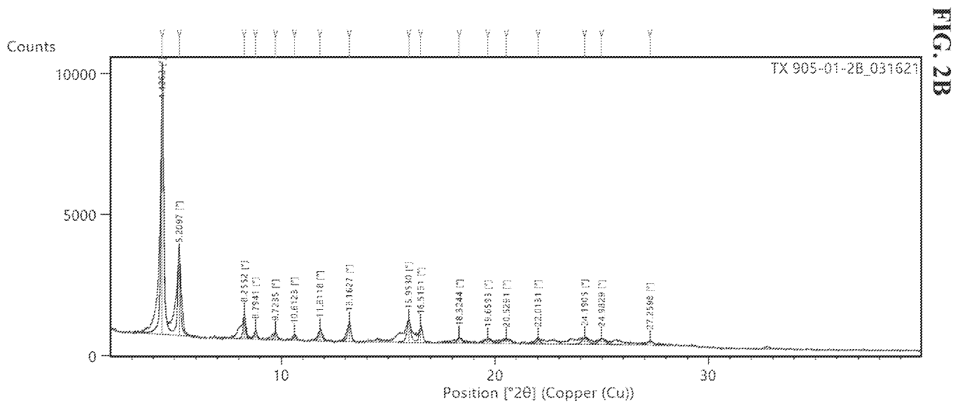

Another embodiment this disclosure as described herein provides a crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile tosylate, i.e., tosylate salt Form A. In various embodiments, tosylate salt Form A has a differential scanning calorimetric (DCS) thermogram. In various embodiments, the DSC thermogram comprises endothermic peaks and exothermic peaks. In some embodiments, tosylate salt Form A has an endothermic DSC peak temperature of about 55° C., e.g., within about ±2% of 55° C. In some embodiments, tosylate salt Form A has an endothermic DSC peak temperature within ±1% of 55° C. or within ±0.5% of 5° C. In some embodiments, tosylate salt Form A has an endothermic DSC peak temperature of about 163° C., e.g., within about ±2% of 163° C. In some embodiments, tosylate salt Form A has an endothermic DSC peak temperature within ±1% of 163° C. or within =0.5% of 163° C. In some embodiments, tosylate salt Form A has an exothermic DSC peak temperature of about 247° C., e.g., within about ±2% of 247° C. In some embodiments, tosylate salt Form A has an exothermic DSC peak temperature within ±1% of 247° C. or within ±0.5% of 247° C. In various embodiments, tosylate salt Form A has as a DSC thermogram with peak temperatures at about 55° C. (e.g., within about ±2% of 55° C., or ±1% of 55° C., or ±0.5% of 55° C.), at about 163° C. (e.g., within about ±2% of 163° C., or ±1% of 163° C., or ±0.5% of 163° C.), and at about 247° C. (e.g., within about ±2% of 247° C., or ±1% of 247° C., or ±0.5% of 247° C.). For example, in some embodiments, tosylate salt Form A has a DSC thermogram substantially shown in FIG. 2A.

In various embodiments, tosylate salt Form A has an X-ray powder diffraction (XRPD) pattern. In some embodiments, tosylate salt Form A has an XRPD pattern comprising a peak at a two-theta angle of 4.4°±0.2°. In some embodiments, tosylate salt Form A has an XRPD pattern comprising a peak at a two-theta angle of 5.2°±0.2°. In some embodiments, tosylate salt Form A has an XRPD pattern comprising a peak at a two-theta angle of 8.2°±0.2°. In some embodiments, tosylate salt Form A has an XRPD pattern comprising a peak at a two-theta angle of 13.1°±0.2°. In some embodiments, tosylate salt Form A has an XRPD pattern comprising a peak at a two-theta angle of 15.9°±0.2°. In some embodiments, tosylate salt Form A has an XRPD pattern comprising a peak at a two-theta angle of 16.5°±0.2°. In some embodiments, tosylate salt Form A has an XRPD pattern comprising peaks at two-theta angles of 4.4°±0.2°, 5.2°±0.2°, 8.2°±0.2°, 13.1°±0.2°, 15.9°±0.2°, and 16.5°±0.2°. For example, in some embodiments, tosylate salt Form A has an XRPD pattern substantially shown in FIG. 2B. In some embodiments as described herein, tosylate salt Form A is a hydrate, solvate, or anhydrate.

In some embodiments as described herein, tosylate salt Form A has a thermal gravimetric analysis (TGA) plot comprising a mass loss of about 2.7% when heated from about 25° C. to about 150° C. For example, in some embodiments, tosylate salt Form A has a TGA plot substantially shown in FIG. 2A.

In some embodiments as described herein, tosylate salt Form A has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile tosylate. In some embodiments, tosylate salt Form A has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile tosylate.

### Glutamate Salt Form A

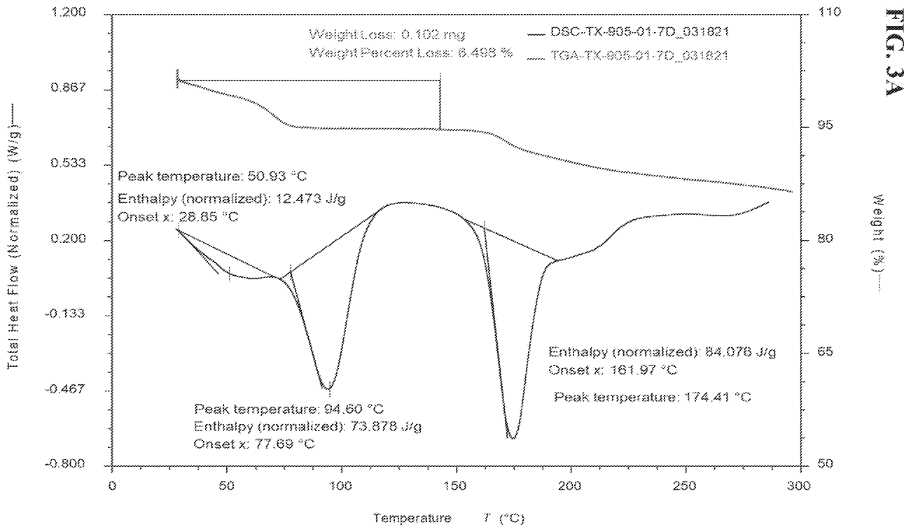

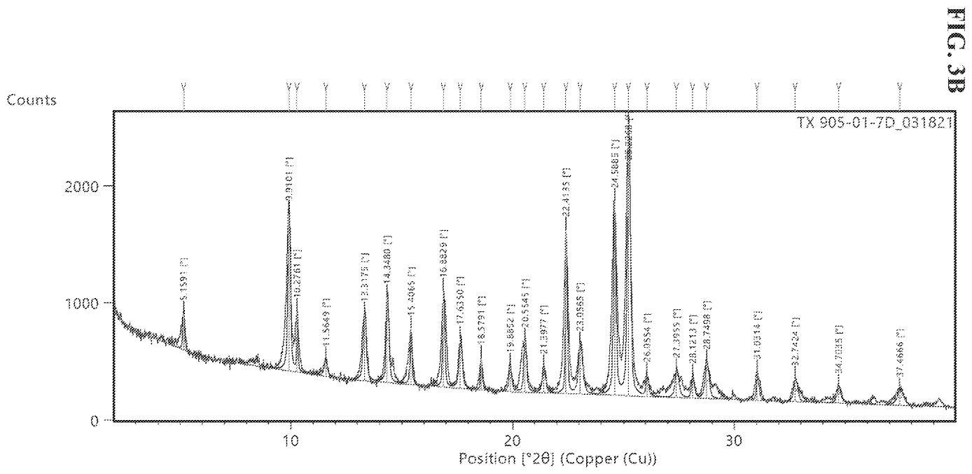

Another embodiment this disclosure as described herein provides a crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glutamate, i.e., glutamate salt Form A. In various embodiments, glutamate salt Form A has a differential scanning calorimetric (DCS) thermogram. In various embodiments, the DSC thermogram comprises endothermic peaks. In some embodiments, glutamate salt Form A has an endothermic DSC peak temperature of about 51° C., e.g., within about ±2% of 51° C. In some embodiments, glutamate salt Form A has an endothermic DSC peak temperature within ±1% of 51° C. or within ±0.5% of 51° C. In some embodiments, glutamate salt Form A has an endothermic DSC peak temperature of about 94° C., e.g., within about ±2% of 94° C. In some embodiments, glutamate salt Form A has an endothermic DSC peak temperature within ±1% of 94° C. or within ±0.5% of 94° C. In some embodiments, glutamate salt Form A has an endothermic DSC peak temperature of about 174° C., e.g., within about ±2% of 174° C. In some embodiments, glutamate salt Form A has an endothermic DSC peak temperature within ±1% of 174° C. or within ±0.5% of 174° C. In various embodiments, glutamate salt Form A has as a DSC thermogram with peak temperatures at about 51° C. (e.g., within about ±2% of 51° C., or ±1% of 51° C., or ±0.5% of 51° C.), at about 94° C. (e.g., within about ±2% of 94° C., or ±1% of 94° C., or ±0.5% of 94° C.), and at about 247° C. (e.g., within about ±2% of 174° C., or ±1% of 174° C., or ±0.5% of 174° C.). For example, in some embodiments, glutamate salt Form A has a DSC thermogram substantially shown in FIG. 3A.

In various embodiments, glutamate salt Form A has an X-ray powder diffraction (XRPD) pattern. In some embodiments, glutamate salt Form A has an XRPD pattern comprising a peak at a two-theta angle of 9.9°±0.2°. In some embodiments, glutamate salt Form A has an XRPD pattern comprising a peak at a two-theta angle of 14.3°±0.2°. In some embodiments, glutamate salt Form A has an XRPD pattern comprising a peak at a two-theta angle of 16.9°±0.2°. In some embodiments, glutamate salt Form A has an XRPD pattern comprising a peak at a two-theta angle of 22.4°±0.2°. In some embodiments, glutamate salt Form A has an XRPD pattern comprising a peak at a two-theta angle of 24.6°±0.2°. In some embodiments, glutamate salt Form A has an XRPD pattern comprising a peak at a two-theta angle of 25.2°±0.2°. In some embodiments, glutamate salt Form A has an XRPD pattern comprising peaks at two-theta angles of 9.9°±0.2°, 14.3°±0.2°, 16.9°±0.2°, 22.4°±0.2°, 24.6°±0.2°, and 25.2°±0.2°. For example, in some embodiments, glutamate salt Form A has an XRPD pattern substantially shown in FIG. 3B. In some embodiments as described herein, glutamate salt Form A is a hydrate or solvate.

In some embodiments as described herein, glutamate salt Form A has a thermal gravimetric analysis (TGA) plot comprising a mass loss of about 6.5% when heated from about 25° C. to about 150° C. For example, in some embodiments, glutamate salt Form A has a TGA plot substantially shown in FIG. 3A.

In some embodiments as described herein, glutamate salt Form A has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glutamate. In some embodiments, glutamate salt Form A has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glutamate.

### Fumarate Salt Form A

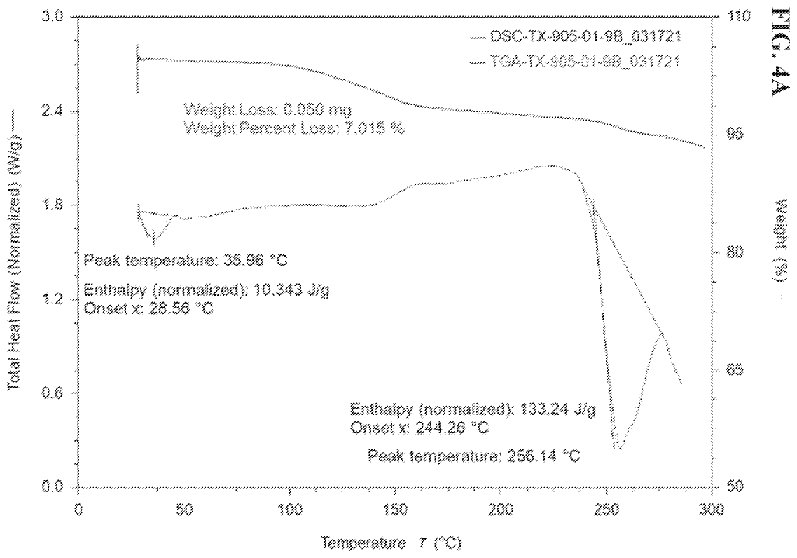

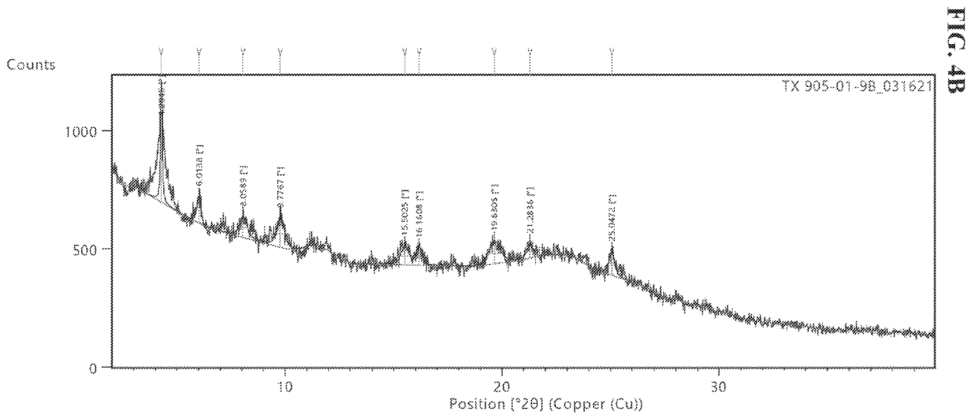

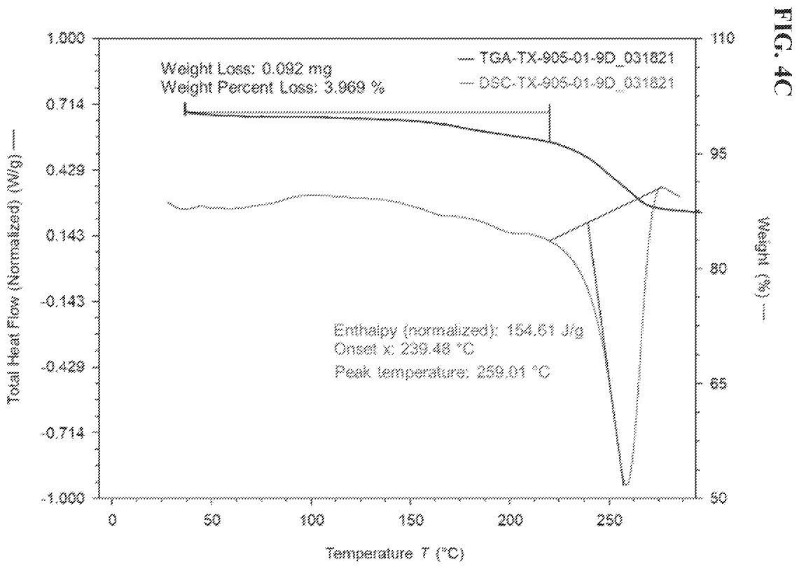

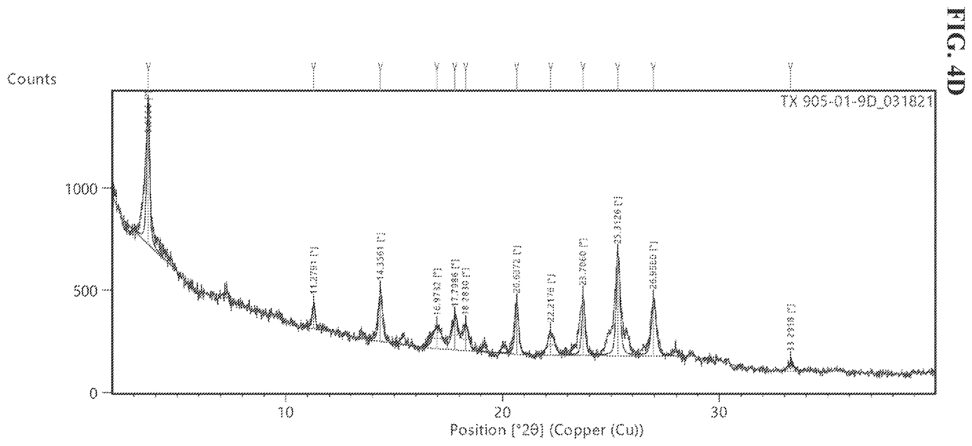

Another embodiment this disclosure as described herein provides a crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile fumarate, i.e., fumarate salt Form A. In various embodiments, fumarate salt Form A has a differential scanning calorimetric (DCS) thermogram. In various embodiments, the DSC thermogram comprises endothermic peaks. In some embodiments, fumarate salt Form A has an endothermic DSC peak temperature of about 36° C., e.g., within about ±2% of 36° C. In some embodiments, fumarate salt Form A has an endothermic DSC peak temperature within ±1% of 36° C. or within ±0.5% of 36° C. In some embodiments, fumarate salt Form A has an endothermic DSC peak temperature of about 256° C., e.g., within about ±2% of 256° C. In some embodiments, fumarate salt Form A has an endothermic DSC peak temperature within ±1% of 256° C. or within ±0.5% of 256° C. In various embodiments, fumarate salt Form A has as a DSC thermogram with peak temperatures at about 36° C. (e.g., within about ±2% of 36° C., or ±1% of 36° C., or ±0.5% of 36° C.) and at about 256° C. (e.g., within about ±2% of 256° C., or ±1% of 256° C., or ±0.5% of 256° C.). For example, in some embodiments, fumarate salt Form A has a DSC thermogram substantially shown in FIG. 4A.

In various embodiments, fumarate salt Form A has an X-ray powder diffraction (XRPD) pattern. In some embodiments, fumarate salt Form A has an XRPD pattern comprising a peak at a two-theta angle of 4.3°±0.2°. In some embodiments, fumarate salt Form A has an XRPD pattern comprising a peak at a two-theta angle of 6.0°±0.2°. In some embodiments, fumarate salt Form A has an XRPD pattern comprising a peak at a two-theta angle of 9.8°±0.2°. In some embodiments, fumarate salt Form A has an XRPD pattern comprising a peak at a two-theta angle of 19.6°±0.2°. In some embodiments, fumarate salt Form A has an XRPD pattern comprising a peak at a two-theta angle of 25.0°±0.2°. In some embodiments, fumarate salt Form A has an XRPD pattern comprising peaks at two-theta angles of 4.3°±0.2°, 6.0°±0.2°, 9.8°±0.2°, 19.6°±0.2°, and 25.0°±0.2°. For example, in some embodiments, fumarate salt Form A has an XRPD pattern substantially shown in FIG. 4B. In some embodiments as described herein, fumarate salt Form A is a hydrate or solvate.

In some embodiments as described herein, fumarate salt Form A has a thermal gravimetric analysis (TGA) plot comprising a mass loss of about 7.0% when heated from about 25° C. to about 150° C. For example, in some embodiments, fumarate salt Form A has a TGA plot substantially shown in FIG. 4A.

In some embodiments as described herein, fumarate salt Form A has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile fumarate. In some embodiments, fumarate salt Form A has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile fumarate.

### Fumarate Salt Form B

Another embodiment this disclosure as described herein provides a crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile fumarate, i.e., fumarate salt Form B. In various embodiments, fumarate salt Form B has a differential scanning calorimetric (DCS) thermogram. In various embodiments, the DSC thermogram comprises endothermic peaks. In some embodiments, fumarate salt Form B has an endothermic DSC peak temperature of about 259° C., e.g., within about ±2% of 269° C. In some embodiments, fumarate salt Form B has an endothermic DSC peak temperature within ±1% of 259° C. or within ±0.5% of 259° C. For example, in some embodiments, fumarate salt Form B has a DSC thermogram substantially shown in FIG. 4C.

In various embodiments, fumarate salt Form B has an X-ray powder diffraction (XRPD) pattern. In some embodiments, fumarate salt Form B has an XRPD pattern comprising a peak at a two-theta angle of 3.6°±0.2°. In some embodiments, fumarate salt Form B has an XRPD pattern comprising a peak at a two-theta angle of 14.3°±0.2°. In some embodiments, fumarate salt Form B has an XRPD pattern comprising a peak at a two-theta angle of 20.6°±0.2°. In some embodiments, fumarate salt Form B has an XRPD pattern comprising a peak at a two-theta angle of 23.7°±0.2°. In some embodiments, fumarate salt Form B has an XRPD pattern comprising a peak at a two-theta angle of 25.3°±0.2°. In some embodiments, fumarate salt Form B has an XRPD pattern comprising a peak at a two-theta angle of 26.9°±0.2°. In some embodiments, fumarate salt Form B has an XRPD pattern comprising peaks at two-theta angles of 3.6°±0.2°, 14.3°±0.2°, 20.6°±0.2°, 23.7°±0.2°, 25.3°±0.2°, and 26.9°±0.2°. For example, in some embodiments, fumarate salt Form B has an XRPD pattern substantially shown in FIG. 4D. In some embodiments as described herein, fumarate salt Form B is a hydrate or solvate.

In some embodiments as described herein, fumarate salt Form B has a thermal gravimetric analysis (TGA) plot comprising a mass loss of about 3.9% when heated from about 25° C. to about 150° C. For example, in some embodiments, fumarate salt Form B has a TGA plot substantially shown in FIG. 4C.

In some embodiments as described herein, fumarate salt Form B has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile fumarate. In some embodiments, fumarate salt Form B has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile fumarate.

### Glycolate Salt Form A

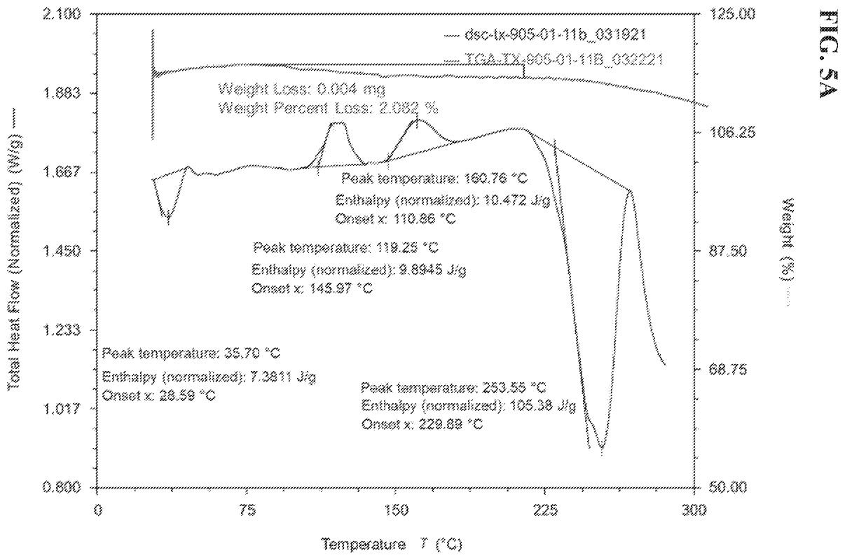

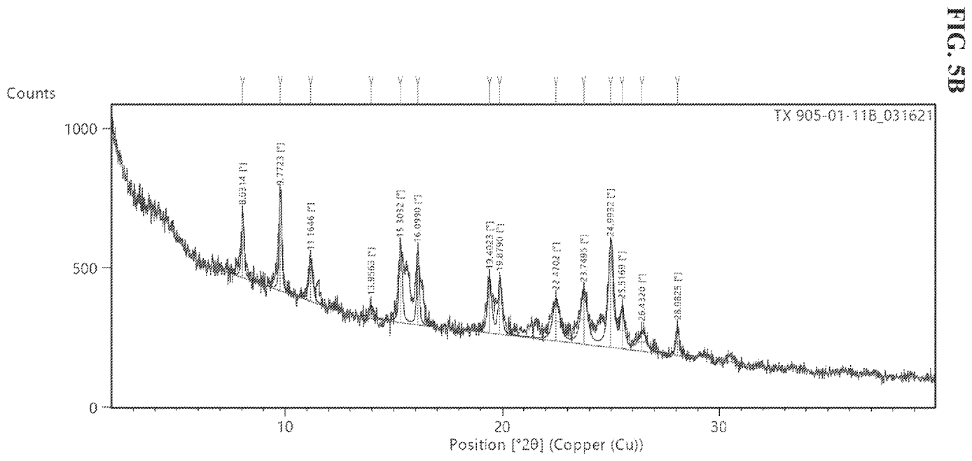

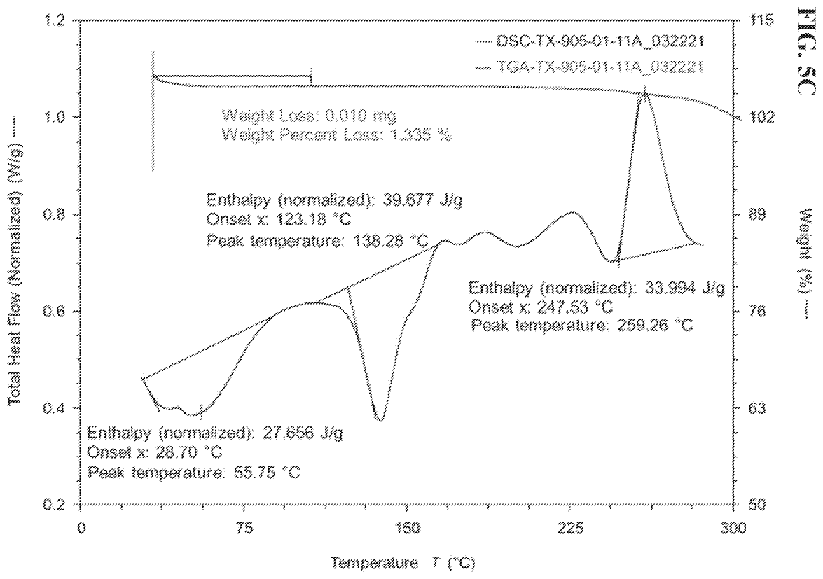

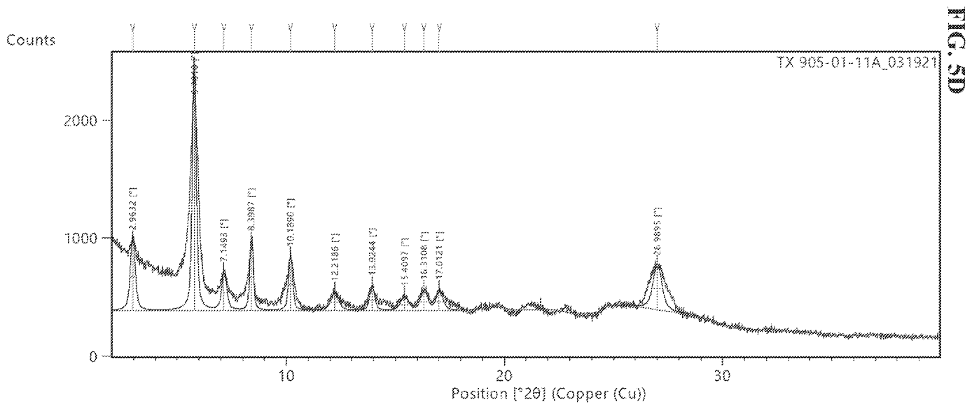

Another embodiment this disclosure as described herein provides a crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glycolate, i.e., glycolate salt Form A. In various embodiments, glycolate salt Form A has a differential scanning calorimetric (DCS) thermogram. In various embodiments, the DSC thermogram comprises endothermic peaks and exothermic peaks. In some embodiments, glycolate salt Form A has an endothermic DSC peak temperature of about 36° C., e.g., within about ±2% of 36° C. In some embodiments, glycolate salt Form A has an endothermic DSC peak temperature within ±1% of 36° C. or within ±0.5% of 36° C. In some embodiments, glycolate salt Form A has an exothermic DSC peak temperature of about 119° C., e.g., within about ±2% of 119° C. In some embodiments, glycolate salt Form A has an exothermic DSC peak temperature within ±1% of 119° C. or within ±0.5% of 119° C. In some embodiments, glycolate salt Form A has an exothermic DSC peak temperature of about 161° C., e.g., within about ±2% of 161° C. In some embodiments, glycolate salt Form A has an exothermic DSC peak temperature within ±1% of 161° C. or within ±0.5% of 161° C. In some embodiments, glycolate salt Form A has an endothermic DSC peak temperature of about 253° C., e.g., within about ±2% of 253° C. In some embodiments, glycolate salt Form A has an endothermic DSC peak temperature within ±1% of 253° C. or within ±0.5% of 253° C. In various embodiments, glycolate salt Form A has as a DSC thermogram with peak temperatures at about 36° C. (e.g., within about ±2% of 36° C., or ±1% of 36° C., or ±0.5% of 36° C.), at about 119° C. (e.g., within about ±2% of 119° C., or ±1% of 119° C., or ±0.5% of 119° C.), at about 161° C. (e.g., within about ±2% of 161° C., or ±1% of 161° C., or ±0.5% of 161° C.), and at about 253° C. (e.g., within about ±2% of 253° C., or ±1% of 253° C., or ±0.5% of 253° C.). For example, in some embodiments, fumarate salt Form A has a DSC thermogram substantially shown in FIG. 5A.

In various embodiments, glycolate salt Form A has an X-ray powder diffraction (XRPD) pattern. In some embodiments, glycolate salt Form A has an XRPD pattern comprising a peak at a two-theta angle of 9.8°±0.2°. In some embodiments, glycolate salt Form A has an XRPD pattern comprising a peak at a two-theta angle of 15.3°±0.2°. In some embodiments, glycolate salt Form A has an XRPD pattern comprising a peak at a two-theta angle of 16.1°±0.2°. In some embodiments, glycolate salt Form A has an XRPD pattern comprising a peak at a two-theta angle of 25.0°±0.2°. In some embodiments, glycolate salt Form A has an XRPD pattern comprising peaks at two-theta angles of 9.8°±0.2°, 15.3°±0.2°, 16.1°±0.2°, and 25.0°±0.2°. For example, in some embodiments, glycolate salt Form A has an XRPD pattern substantially shown in FIG. 5B. In some embodiments as described herein, glycolate salt Form A is a hydrate or solvate.

In some embodiments as described herein, glycolate salt Form A has a thermal gravimetric analysis (TGA) plot comprising a mass loss of about 2.1% when heated from about 25° C. to about 150° C. For example, in some embodiments, glycolate salt Form A has a TGA plot substantially shown in FIG. 5A.

In some embodiments as described herein, glycolate salt Form A has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glycolate. In some embodiments, glycolate salt Form A has a purity of at least 98% by weight of 2-(4-(4 -(aminomethyl)-1-oxo-1,2-dihydrophthalazin-6-yl)-1-methyl-1H-pyrazol-5-yl)-4-chloro-6-cyclopropoxy-3-fluorobenzonitrile glycolate.

### Glycolate Salt Form B

Another embodiment this disclosure as described herein provides a crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glycolate, i.e., glycolate salt Form B. In various embodiments, glycolate salt Form B has a differential scanning calorimetric (DCS) thermogram. In various embodiments, the DSC thermogram comprises endothermic peaks and exothermic peaks. In some embodiments, glycolate salt Form B has an endothermic DSC peak temperature of about 56° C., e.g., within about ±2% of 56° C. In some embodiments, glycolate salt Form B has an endothermic DSC peak temperature within ±1% of 56° C. or within ±0.5% of 56° C. In some embodiments, glycolate salt Form B has an endothermic DSC peak temperature of about 138° C., e.g., within about ±2% of 138° C. In some embodiments, glycolate salt Form B has an endothermic DSC peak temperature within ±1% of 138° C. or within ±0.5% of 138° C. In some embodiments, glycolate salt Form B has an exothermic DSC peak temperature of about 259° C., e.g., within about ±2% of 259° C. In some embodiments, glycolate salt Form B has an exothermic DSC peak temperature within ±1% of 259° C. or within ±0.5% of 259° C. In various embodiments, glycolate salt Form B has as a DSC thermogram with peak temperatures at about 56° C. (e.g., within about ±2% of 56° C., or ±1% of 56° C., or ±0.5% of 56° C.), at about 138° C. (e.g., within about ±2% of 138° C., or ±1% of 138° C., or ±0.5% of 138° C.), and at about 259° C. (e.g., within about ±2% of 259° C., or ±1% of 259° C., or ±0.5% of 259° C.). For example, in some embodiments, glycolate salt Form B has a DSC thermogram substantially shown in FIG. 5C.

In various embodiments, glycolate salt Form B has an X-ray powder diffraction (XRPD) pattern. In some embodiments, glycolate salt Form B has an XRPD pattern comprising a peak at a two-theta angle of 2.9°±0.2°. In some embodiments, glycolate salt Form B has an XRPD pattern comprising a peak at a two-theta angle of 5.8°±0.2°. In some embodiments, glycolate salt Form B has an XRPD pattern comprising a peak at a two-theta angle of 8.4°±0.2°. In some embodiments, glycolate salt Form B has an XRPD pattern comprising a peak at a two-theta angle of 10.2°±0.2°. In some embodiments, glycolate salt Form B has an XRPD pattern comprising a peak at a two-theta angle of 27.0°±0.2°. In some embodiments, glycolate salt Form A has an XRPD pattern comprising peaks at two-theta angles of 2.9°±0.2°, 5.8°±0.2°, 8.4°±0.2°, 10.2°±0.2°, and 27.0°±0.2°. For example, in some embodiments, glycolate salt Form B has an XRPD pattern substantially shown in FIG. 5D. In some embodiments as described herein, glycolate salt Form B is a hydrate, solvate, or anhydrate.

In some embodiments as described herein, glycolate salt Form B has a thermal gravimetric analysis (TGA) plot comprising a mass loss of about 1.3% when heated from about 25° C. to about 150° C. For example, in some embodiments, glycolate salt Form B has a TGA plot substantially shown in FIG. 5C.

In some embodiments as described herein, glycolate salt Form B has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glycolate. In some embodiments, glycolate salt Form B has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glycolate.

### Hippurate Salt Form A

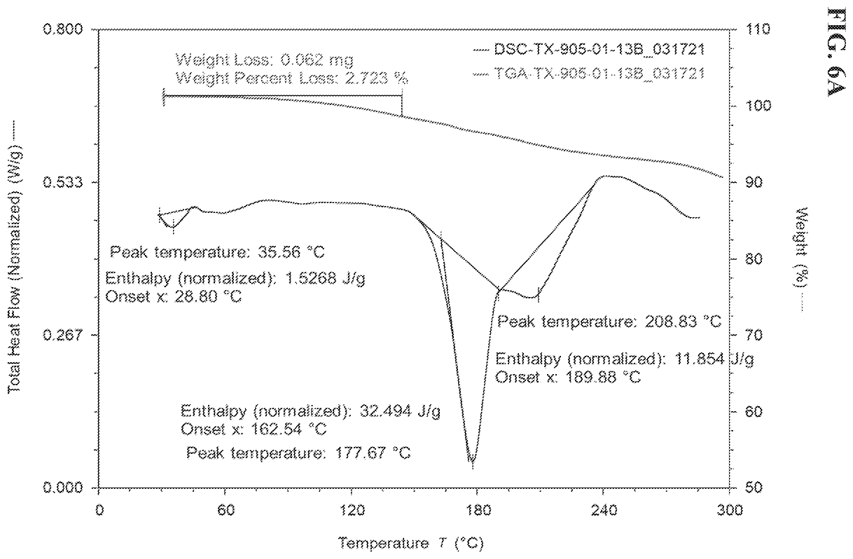

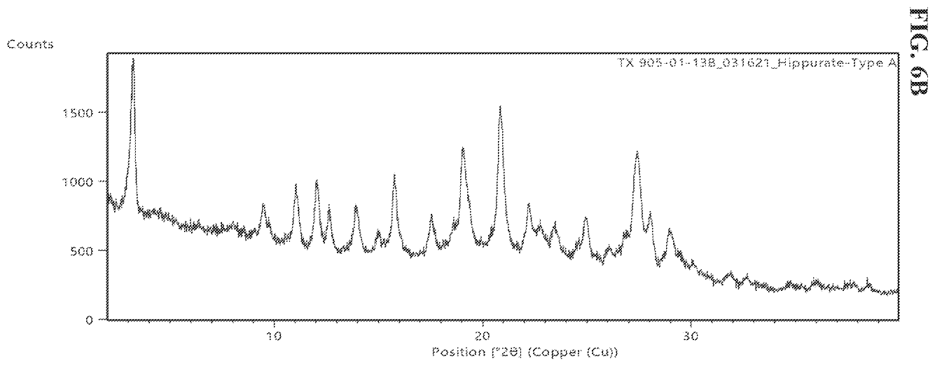

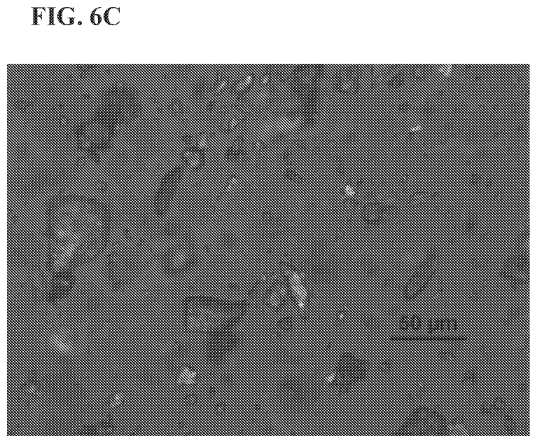

Another embodiment this disclosure as described herein provides a crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hippurate, i.e., hippurate salt Form A. In various embodiments, hippurate salt Form A has a differential scanning calorimetric (DCS) thermogram. In various embodiments, the DSC thermogram comprises endothermic peaks. In some embodiments, hippurate salt Form A has an endothermic DSC peak temperature of about 36° C., e.g., within about ±2% of 36° C. In some embodiments, hippurate salt Form A has an endothermic DSC peak temperature within ±1% of 36° C. or within ±0.5% of 36° C. In some embodiments, hippurate salt Form A has an endothermic DSC peak temperature of about 177° C., e.g., within about ±2% of 177° C. In some embodiments, hippurate salt Form A has an endothermic DSC peak temperature within ±1% of 177° C. or within ±0.5% of 177° C. In some embodiments, hippurate salt Form A has an endothermic DSC peak temperature of about 208° C., e.g., within about ±2% of 208° C. In some embodiments, hippurate salt Form A has an endothermic DSC peak temperature within ±1% of 208° C. or within ±0.5% of 208° C. In various embodiments, hippurate salt Form A has as a DSC thermogram with peak temperatures at about 36° C. (e.g., within about ±2% of 36° C., or #1% of 36° C., or ±0.5% of 36° C.), at about 177° C. (e.g., within about ±2% of 177° C., or ±1% of 177° C., or ±0.5% of 177° C.), and at about 208° C. (e.g., within about ±2% of 208° C., or ±1% of 208° C., or ±0.5% of 208° C.). For example, in some embodiments, hippurate salt Form A has a DSC thermogram substantially shown in FIG. 6A.

In various embodiments, hippurate Form A has an X-ray powder diffraction (XRPD) pattern. In some embodiments, hippurate Form A has an XRPD pattern comprising a peak at a two-theta angle of 3.2°±0.2°. In some embodiments, hippurate Form A has an XRPD pattern comprising a peak at a two-theta angle of 19.1°±0.2°. In some embodiments, hippurate Form A has an XRPD pattern comprising a peak at a two-theta angle of 20.8°±0.2°. In some embodiments, hippurate Form A has an XRPD pattern comprising a peak at a two-theta angle of 27.4°±0.2°. In some embodiments, hippurate Form A has an XRPD pattern comprising peaks at two-theta angles of 3.2°±0.2°, 19.1°±0.2°, 20.8°±0.2°, and 27.4°±0.2°. For example, in some embodiments, hippurate Form A has an XRPD pattern substantially shown in FIG. 6B. In some embodiments as described herein, hippurate Form B is a hydrate, solvate, or anhydrate.

In some embodiments as described herein, hippurate salt Form A has a thermal gravimetric analysis (TGA) plot comprising a mass loss of about 2.7% when heated from about 25° C. to about 150° C. For example, in some embodiments, hippurate salt Form A has a TGA plot substantially shown in FIG. 6A.

In some embodiments as described herein, hippurate salt Form A has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hippurate. In some embodiments, hippurate salt Form A has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hippurate.

### Phosphate Salt Form A

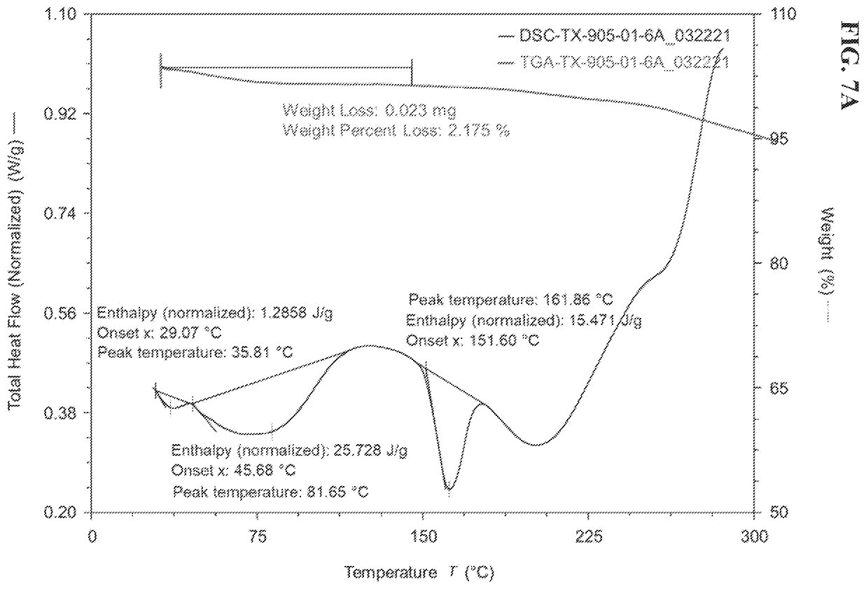

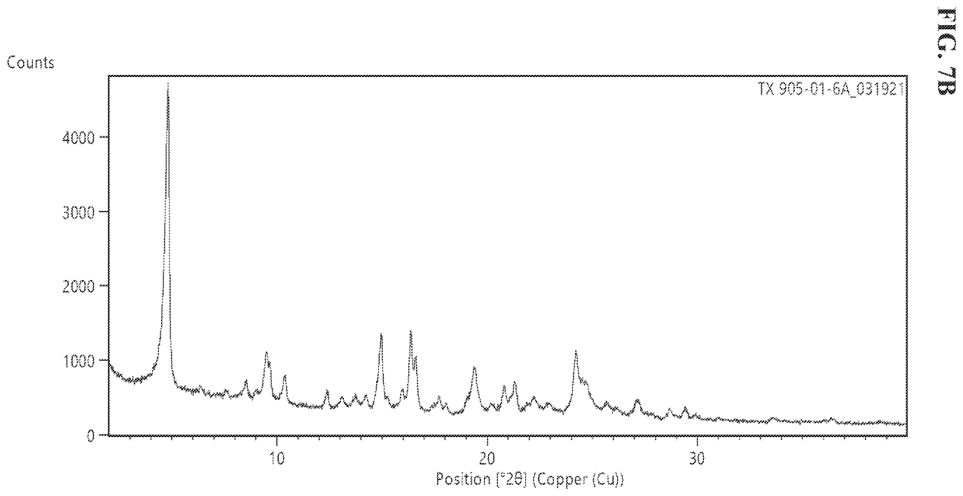

Another embodiment this disclosure as described herein provides a crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile phosphate, i.e., phosphate salt Form A. In various embodiments, phosphate salt Form A has a differential scanning calorimetric (DCS) thermogram. In various embodiments, the DSC thermogram comprises endothermic peaks. In some embodiments, phosphate salt Form A has an endothermic DSC peak temperature of about 36° C., e.g., within about ±2% of 36° C. In some embodiments, phosphate salt Form A has an endothermic DSC peak temperature within ±1% of 36° C. or within ±0.5% of 36° C. In some embodiments, phosphate salt Form A has an endothermic DSC peak temperature of about 82° C., e.g., within about ±2% of 82° C. In some embodiments, phosphate salt Form A has an endothermic DSC peak temperature within ±1% of 82° C. or within ±0.5% of 82° C. In some embodiments, phosphate salt Form A has an endothermic DSC peak temperature of about 162° C., e.g., within about ±2% of 162° C. In some embodiments, phosphate salt Form A has an endothermic DSC peak temperature within ±1% of 162° C. or within ±0.5% of 162° C. In various embodiments, phosphate salt Form A has as a DSC thermogram with peak temperatures at about 36° C. (e.g., within about ±2% of 36° C., or ±1% of 36° C., or ±0.5% of 36° C.), at about 82° C. (e.g., within about ±2% of 82° C., or ±1% of 82° C., or ±0.5% of 82° C.), and at about 162° C. (e.g., within about ±2% of 162° C., or ±1% of 162° C., or ±0.5% of 162° C.). For example, in some embodiments, phosphate salt Form A has a DSC thermogram substantially shown in FIG. 7A.

In various embodiments, phosphate salt Form A has an X-ray powder diffraction (XRPD) pattern. In some embodiments, phosphate salt Form A has an XRPD pattern comprising a peak at a two-theta angle of 6.6°±0.2°. In some embodiments, phosphate salt Form A has an XRPD pattern comprising a peak at a two-theta angle of 17.0°±0.2°. In some embodiments, phosphate salt Form A has an XRPD pattern comprising a peak at a two-theta angle of 18.9°±0.2°. In some embodiments, phosphate salt Form A has an XRPD pattern comprising a peak at a two-theta angle of 20.4°±0.2°. In some embodiments, phosphate salt Form A has an XRPD pattern comprising a peak at a two-theta angle of 22.9°±0.2°. In some embodiments, phosphate salt Form A has an XRPD pattern comprising peaks at two-theta angles of 6.6°±0.2°, 17.0°±0.2°, 18.9°±0.2°, 20.4°±0.2°, and 22.9°±0.2°. For example, in some embodiments, phosphate salt Form A has an XRPD pattern substantially shown in FIG. 7B. In some embodiments as described herein, phosphate salt Form A is a hydrate or solvate.

In some embodiments as described herein, phosphate salt Form A has a thermal gravimetric analysis (TGA) plot comprising a mass loss of about 2.2% when heated from about 25° C. to about 150° C. For example, in some embodiments, phosphate salt Form A has a TGA plot substantially shown in FIG. 7A.

In some embodiments as described herein, phosphate salt Form A has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile phosphate. In some embodiments, phosphate salt Form A has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile phosphate.

### Processes of Preparing Crystalline Forms of Compound 1

In another aspect, this disclosure provides processes of preparing crystalline forms of Compound 1, e.g., sulfate salt Form A, tosylate salt Form A, glutamate salt Form A, fumarate salt Form A, fumarate salt Form B, glycolate salt Form A, glycolate salt Form B, hippurate salt Form A, and phosphate salt Form A. Crystalline forms of sulfate salt Form A, tosylate salt Form A, glutamate salt Form A, fumarate salt Form A, fumarate salt Form B, glycolate salt Form A, glycolate salt Form B, hippurate salt Form A, and phosphate salt Form A can be made by a variety of methods as known to the person of skill in the art and discussed in the Examples below. For example, the crystalline forms as described herein may be prepared by slurry methods.

In various embodiments, the slurry method can be conducted at a variety of temperatures and with a variety of solvents. For example, in some embodiments, the slurry method is conducted at room temperature or at an elevated temperature (e.g., 50° C.). To prepare crystalline forms with the slurry method, amorphous free base of Compound 1 is suspended and stirred in a solvent with the corresponding acid (e.g., sulfuric acid, toluene sulfonic acid, glutamic acid, fumaric acid, glycolic acid, hippuric acid, or phosphoric acid,) at a temperature (e.g., room temperature or elevated temperature) to provide solids. In various embodiments, to provide the crystalline forms as disclosed herein, about 30 mg of amorphous free base of Compound 1 and about 30 mg of the corresponding acid may be combined in about 0.1 to 5 mL of solvent (based on the approximate solubility), with a molar charge ratio of 1:1. The solvent may be selected from methanol (MeOH), tetrahydrofuran (THF), ethyl acetate (EtOAc), or acetone/water (1:1, v/v). The mixture may be stirred (e.g., magnetically) for a time of at least 24 hours (e.g., at least 48 hours, or at least 72 hours, or at least 96 hours). In some embodiment, the mixture is stirred for at least 96 hours (i.e. 4 days) at room temperature. Any precipitate formed may be isolated by centrifugation or other solid-liquid separation techniques known in the art. In some embodiments, if no precipitate is observed after four days at room temperature, the mixture may be stirred at 5° C. for 24 hours. In some embodiments, if still no solids are observed, the mixture may be subjected to slow evaporation at room temperature for four days. In various embodiments, the isolated solid can be air dried at ambient condition.

In some embodiments, to provide sulfate salt Form A, slurry methods may be used. When the slurry method is used, the solvent may be THF and the acid may be sulfuric acid.

In some embodiments, to provide tosylate salt Form A, slurry methods may be used. When the slurry method is used, the solvent may be THF or EtOAc and the acid may be toluene sulfonic acid.

In some embodiments, to provide glutamate salt Form A, slurry methods may be used. When the slurry method is used, the solvent may be acetone/water (1:1, v/v) and the acid may be glutamic acid.

In some embodiments, to provide fumarate salt Form A, slurry methods may be used. When the slurry method is used, the solvent may be THF and the acid may be fumaric acid.

In some embodiments, to provide fumarate salt Form B, slurry methods may be used. When the slurry method is used, the solvent may be acetone/water (1:1, v/v) and the acid may be fumaric acid.

In some embodiments, to provide glycolate salt Form A, slurry methods may be used. When the slurry method is used, the solvent may be THF and the acid may be glycolic acid.

In some embodiments, to provide glycolate salt Form B, slurry methods may be used. When the slurry method is used, the solvent may be MeOH and the acid may be glycolic acid.

In some embodiments, to provide hippurate salt Form A, slurry methods may be used. When the slurry method is used, the solvent may be THF and the acid may be hippuric acid.

In some embodiments, to provide phosphate salt Form A, slurry methods may be used. When the slurry method is used, the solvent may be MeOH and the acid may be phosphoric acid.

### Pharmaceutical Compositions

In another aspect, this disclosure provides pharmaceutical compositions comprising crystalline forms of Compound 1 (e.g., sulfate salt Form A, tosylate salt Form A, glutamate salt Form A, fumarate salt Form A, fumarate salt Form B, glycolate salt Form A, glycolate salt Form B, hippurate salt Form A, and phosphate salt Form A) and an appropriate carrier, excipient or diluent. The exact nature of the carrier, excipient or diluent will depend upon the desired use for the composition, and may range from being suitable or acceptable for veterinary uses to being suitable or acceptable for human use. The composition may optionally include one or more additional compounds. In certain embodiments, the composition may include one or more antibiotic compounds. In another aspect, this disclosure provides pharmaceutical compositions comprising sulfate salt Form A and a pharmaceutically acceptable carrier. In another aspect, this disclosure provides pharmaceutical compositions comprising tosylate salt Form A and a pharmaceutically acceptable carrier. In another aspect, this disclosure provides pharmaceutical compositions comprising glutamate salt Form A and a pharmaceutically acceptable carrier. In another aspect, this disclosure provides pharmaceutical compositions comprising fumarate salt Form A and a pharmaceutically acceptable carrier. In another aspect, this disclosure provides pharmaceutical compositions comprising fumarate salt Form B and a pharmaceutically acceptable carrier. In another aspect, this disclosure provides pharmaceutical compositions comprising glycolate salt Form A and a pharmaceutically acceptable carrier. In another aspect, this disclosure provides pharmaceutical compositions comprising glycolate salt Form B and a pharmaceutically acceptable carrier. In another aspect, this disclosure provides pharmaceutical compositions comprising hippurate salt Form A and a pharmaceutically acceptable carrier. In another aspect, this disclosure provides pharmaceutical compositions comprising phosphate salt Form A and a pharmaceutically acceptable carrier.

When used to treat or prevent such diseases, Compound 1 described herein may be administered singly, as mixtures of one or more compounds or in mixture or combination with other agents useful for treating such diseases and/or the symptoms associated with such diseases. The compounds may also be administered in mixture or in combination with agents useful to treat other disorders or maladies, such as steroids, membrane stabilizers, 5LO inhibitors, leukotriene synthesis and receptor inhibitors, inhibitors of IgE isotype switching or IgE synthesis, IgG isotype switching or IgG synthesis, β-agonists, tryptase inhibitors, aspirin, COX inhibitors, methotrexate, anti-TNF drugs, retuxin, PD4 inhibitors, p38 inhibitors, PDE4 inhibitors, and antihistamines, to name a few. The Compound 1 may be administered in the crystalline forms as described herein, or as pharmaceutical compositions comprising the crystalline forms as described herein.

Pharmaceutical compositions comprising the various crystalline forms of Compound 1 may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

### Methods of Use

The crystalline forms described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

In yet another aspect, the invention provides for methods for inhibiting PRMT5 activity in a cell, comprising contacting the cell in which inhibition of PRMT5 activity is desired in vitro with an effective amount of a crystalline forms of Compound 1 as described herein or pharmaceutical compositions containing the crystalline forms of Compound 1 as described herein. In one embodiment, the cell is an MTAP-deficient cell.

The compositions and methods provided herein are particularly deemed useful for inhibiting PRMT5 activity in a cell in vivo. In one embodiment, a cell in which inhibition of PRMT5 activity is desired is contacted in vivo with a therapeutically effective amount of crystalline forms of Compound 1 as described herein or pharmaceutical compositions containing the crystalline forms of Compound 1 as described herein. In one embodiment, the cell is an MTAP-deficient cell. In one embodiment, the negatively modulating the activity of PRMT5 occurs in the presence of bound MTA.

By negatively modulating the activity of PRMT5, particularly in cases for cells that lack MTAP activity, the methods are designed to inhibit PRMT5 activity to block cellular proliferation. The cells may be contacted in a single dose or multiple doses in accordance with a particular treatment regimen to affect the desired negative modulation of PRMT5. The degree PRMT5 inhibition may be monitored in vitro against the enzyme in the presence and absence of MTA and in the cell using well known methods, including those described in Example B below, to assess the effectiveness of treatment and dosages.

In another aspect, methods of treating cancer comprising administering to a patient having cancer a therapeutically effective amount of crystalline forms of Compound 1 as described herein or pharmaceutical compositions containing the crystalline forms of Compound 1 as described herein. In one embodiment, the cancer is an MTAP-associated cancer.

The compositions and methods provided herein may be used for the treatment of a wide variety of cancer including tumors such as prostate, breast, brain, skin, cervical carcinomas, testicular carcinomas, etc. More particularly, cancers that may be treated by the compositions and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is diffuse large B-cell lymphoma (DLBCL).

In one embodiment, the cancer is an MTAP-associated cancer selected from hepatocellular carcinoma, breast cancer, skin cancer, bladder cancer, liver cancer, pancreatic cancer, and head and neck cancer.

In other embodiments, the cancer is selected from the group consisting of ovarian serous cystadenocarcinoma, squamous cell lung cancer, lung adenocarcinoma, mesothelioma; esophageal squamous cell carcinoma, gastric adenocarcinoma, pancreatic ductal adenocarcinoma, kidney adenocarcinoma, bladder transitional cell carcinoma, hepatocellular carcinoma, cholangiocarcinoma, cholangiocarcinoma; osteosarcoma, multiple myeloma, astrocytoma, glioma, glioblastoma, uterine sarcoma, acute myeloid leukemia, acute lymphoblastic leukemia, non-Hodgkin's lymphoma, malignant melanoma, endometrial carcinoma and thyroid carcinoma.

In other embodiments, the cancer is selected from the group consisting of bladder cancer, bone cancer, brain cancer, blood cancer, breast cancer, colorectal cancer, esophageal cancer, gastric cancer, head and neck cancer, kidney cancer, liver cancer, lung cancer, mesothelioma, ovarian cancer, pancreatic cancer, skin cancer, thyroid cancer and uterine cancer.

The concentration and route of administration to the patient will vary depending on the cancer to be treated. The crystalline forms of Compound 1 as described herein or pharmaceutical compositions containing the crystalline forms of Compound 1 as described herein also may be co-administered with other anti-neoplastic compounds, e.g., chemotherapy, or used in combination with other treatments, such as radiation or surgical intervention, either as an adjuvant prior to surgery or post-operatively.

### EXAMPLES

The following Examples are intended to illustrate further certain embodiments of the invention and are not intended to limit the scope of the invention.

### Example 1: Salt Break of Compound 1

Compound 1 can be prepared as a gum according to procedures disclosed in published International Application No. WO2021050915. See Example 16-8.

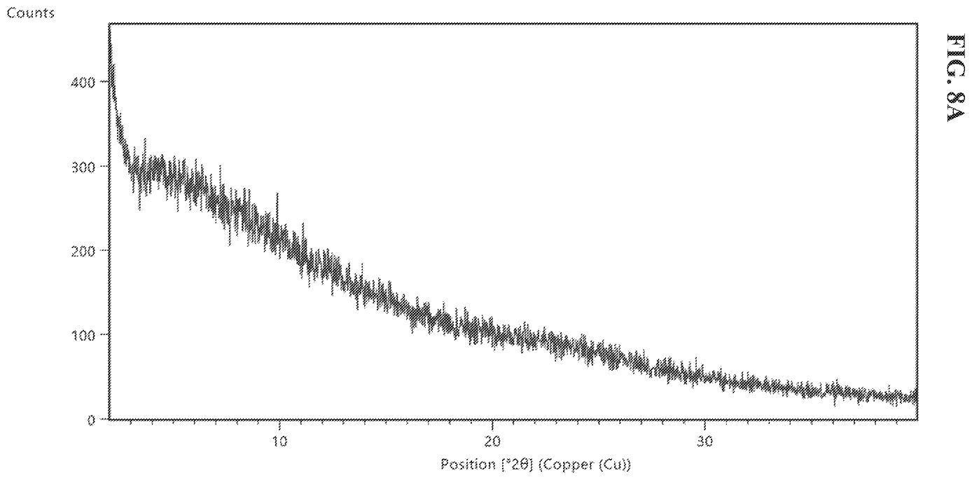

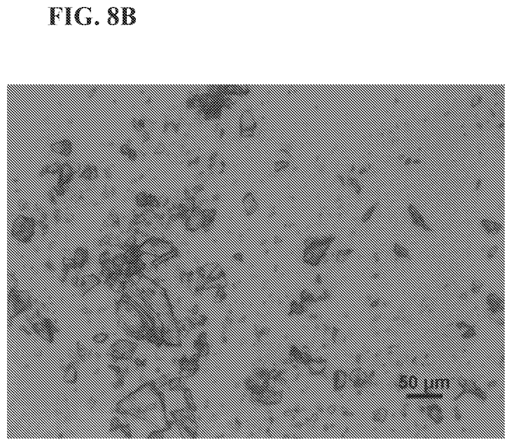

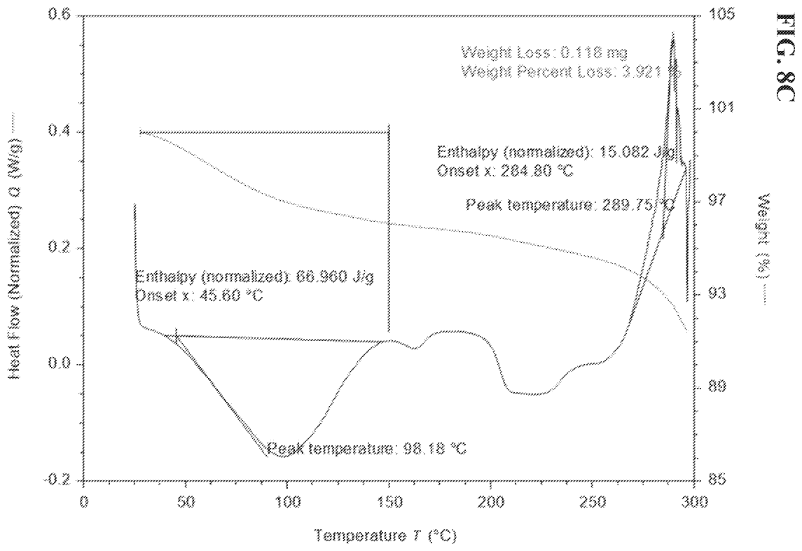

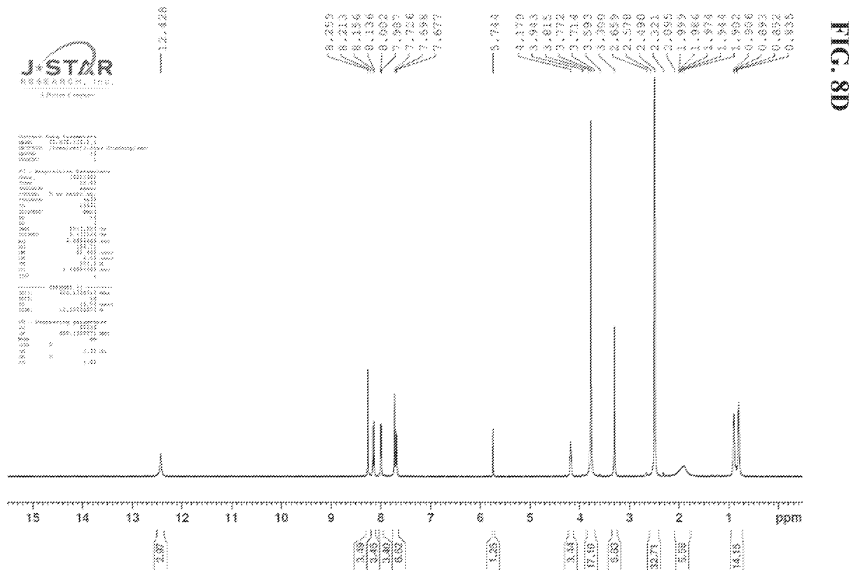

An amorphous HCl salt of Compound 1 was used in a salt breaking experiment at 500 mg scale. In a 500 mL glass, 502 mg of the HCl salt was added with 41.7 mL of Milli-Q water. The mixture was stirred to provide a uniform solution. To the solution, 3.3 mL of NaHCO₃ (saturated solution) was added and the pH was measured to be 7.28. To the solution, 50 mL of dichloromethane (DCM) was added with continuous stirring for 30 minutes. The mixture was then transferred to a 1 L separating funnel and two clear phases separated out within 10 min. The DCM layer was isolated in a 250 mL round bottom flaks and subject to evaporation at 40° C. The isolated solids were dried at 25° C. under vacuum overnight. A yield of 95% was obtained (calculated based on theoretical weight of free base). The solids were shown to be amorphous by XRD (FIG. 8A). A polarized light microscopy image in FIG. 8B shows the solids obtained. Additionally, the solids were characterized by DSC and TGA, as shown in FIG. 8C. The solids were confirmed to be free base with residual DCM by NMR (FIG. 8D).

### Example 2: Salt Screening of Compound 1

Using the amorphous free base from Example 1, 64 salt screen experiments were conducted using 16 acids in four solvent systems, plus four blank experiments in each respective solvent system. Slurry methods as disclosed herein were used to provide salt polymorphs of Compound 1. For each experiment, about 30 mg of amorphous free base form Example 1 and about 30 mg of a corresponding acid (e.g., sulfate, tosylate, glutamate, fumarate, glycolate, hippurate, or phosphate) were mixed in 0.3-4.4 mL solvent (based on the approximate solubility), with a molar charge ratio of 1:1. After magnetically stirring for four days at room temperature, any precipitation was isolated by centrifugation. If there was no precipitation after four days at room temperature, the clear solutions were transferred to stir at 5° C. to induce crystallization for 24 hours. If still no solids were observed, the final clear solution was subjected to slow evaporation at room temperature for four days. Any isolated solid was air dried at ambient condition before analysis. Table 1 summarizes the experiments conducted and the resulting crystal forms obtained.

TABLE 1

| | | Solvent | | | | |
|---|---|---|---|---|---|---|
| | | | | | | Acetone/H₂O |
| Acid | | pKa | MeOH | THF | EtOAc | (1:1, v/v) |
| 0 | Blank | | No solid | Amorphous | Amorphous | No solid isolated |
| | | | isolated | | | |
| 1 | Sulfuric acid | −3 | No solid | Sulfate Salt | No solid | No solid isolated |
| | | | isolated | Form A | isolated | |
| 2 | Toluene | 1.34 | No solid | Tosylate Salt | Tosylate Salt | No solid isolated |
| | Sulfonic | | isolated | Form A | Form A | |
| | acid | | | | | |
| 3 | Oxalic acid | 1.27 | No solid | Amorphous | Amorphous | No solid isolated |
| | | | isolated | | | |
| 4 | L-Aspartic | 1.88 | No solid | Aspartic | Aspartic | Poorly |
| | acid | | isolated | acid + extra | acid + extra | crystalline |
| | | | | peaks | peaks | |
| 5 | Maleic acid | 1.92 | No solid | No solid | Amorphous | No solid isolated |
| | | | isolated | isolated | | |
| 6 | Phosphoric | 1.96 | Phosphate | No solid | Amorphous | No solid isolated |
| | acid | | Salt Form | isolated | | |
| | | | A | | | |
| 7 | Glutamic | 2.19 | Glutamic | Glutamic | Glutamic | Glutamate Salt |
| | acid | | acid | acid + extra | acid + extra | Form A |
| | | | | peaks | peaks | |
| 8 | Tartaric acid | 3.02 | No solid | Amorphous | No solid | No solid isolated |
| | | | isolated | | isolated | |
| 9 | Fumaric | 3.03 | No solid | Fumarate Salt | Amorphous | Fumarate Salt |
| | acid | | isolated | Form A | | Form B |
| 10 | Citric acid | 3.13 | No solid | No solid | Amorphous | No solid isolated |
| | | | isolated | isolated | | |
| 11 | Glycolic | 3.28 | Glycolate | Glycolate | Glycolate | No solid isolated |
| | acid | | Salt Form | Salt Form A | Salt Form A | |
| | | | B | | | |
| 12 | Malic acid | 3.16 | Amorphous | Amorphous | Amorphous | No solid isolated |
| 13 | Hippuric | 3.55 | No solid | Hippurate | Amorphous | Hippurate Salt |
| | acid | | isolated | Salt Form | | Form A + |
| | | | | A | | extra peaks |
| 14 | Gluconic | 3.76 | No solid | Amorphous | Amorphous | No solid isolated |
| | acid | | isolated | | | |
| 15 | Lactic acid | 3.86 | No solid | No solid | No solid | No solid isolated |
| | | | isolated | isolated | isolated | |
| 16 | Ascorbic | 4.17 | Amorphous | No solid | Amorphous | Amorphous |
| | acid | | | isolated | | |

From Table 1, it can be seen that nine crystalline salts were obtained: sulfate salt Form A, tosylate salt Form A, glutamate salt Form A, fumarate salt Form A, fumarate salt Form B, glycolate salt Form A, glycolate salt Form B, hippurate salt Form A, and phosphate salt Form A. Solids were successfully isolated in 22 other conditions, however, the solids were characterized to be either amorphous, poorly crystalline, or counterions themselves. The following examples discuss the characterization of the nine crystalline salts.

### Example 3: Characterization of Crystalline Sulfate Salt Form A of Compound 1

As shown in Table 1, sulfate salt Form A of Compound 1 was obtained with slurry methods described in Example 2 from a mixture of the free base of Compound 1, sulfuric acid, and THF. The resulting solid remained a wet paste, likely due to hygroscopicity after air drying. As such, DSC/TGA data were not collected. FIG. 1A shows and XRD of the sulfate salt Form A crystalline form of Compound 1.

Table 2 describes the XRD pattern of sulfate salt Form A crystalline form of Compound 1 shown in FIG. 1A.

TABLE 2

| Pos. (°2θ) | Intensity | Height (cts) |
|---|---|---|
| 6.6113 | High | 1001.96 |
| 10.9662 | Medium | 339.58 |
| 11.6831 | Low | 81.7 |
| 12.879 | Low | 113.03 |
| 14.791 | Low | 110.71 |
| 16.4822 | Low | 279.99 |
| 17.0296 | Medium | 354.94 |
| 17.9287 | Low | 178.07 |
| 18.8999 | Medium | 393.81 |
| 19.8809 | Low | 317.09 |
| 20.3998 | Medium | 570.12 |
| 22.9003 | High | 2588.56 |
| 24.7426 | Low | 210.43 |
| 26.0253 | Low | 168.45 |
| 26.5477 | Low | 143.82 |
| 28.5068 | Low | 230.27 |
| 29.2913 | Low | 305.63 |
| 31.4136 | Low | 70.04 |

### Example 4: Characterization of Crystalline Tosylate Salt Form A of Compound 1

As shown in Table 1, tosylate salt Form A of Compound 1 was obtained with the slurry methods as described in Example 2 from a mixture of the free base of Compound 1, toluene sulfonic acid, and either THF or EtOAc. The solids were characterized by DSC/TGA and XRPD, as shown in FIGS. 2A and 2B, respectively. Table 3 summarizes the characterization of tosylate salt Form A of Compound 1.

TABLE 3

| | |
|---|---|
| Sample | Tosylate Salt Form A |
| Crystallinity by XRPD | Medium |
| Wt. Loss in TGA (%, ° C.) | 2.66 (150) |
| Endotherm in DSC (peak, ° C.) | 55.62 |
| | 163.74 |
| Exotherm in DSC (peak, ° C.) | 247.71 |
| Form | Hydrate/solvate or anhydrate |

Table 4 describes the XRD pattern of tosylate salt Form A crystalline form of Compound 1 shown in FIG. 2B.

TABLE 4

| Pos. (°2θ) | Intensity | Height (cts) |
|---|---|---|
| 4.4262 | High | 8943.79 |
| 5.2097 | High | 2908.2 |
| 8.2552 | Medium | 871.11 |
| 8.7941 | Low | 284.88 |
| 9.7235 | Low | 238.44 |
| 10.6123 | Low | 157.3 |
| 11.8118 | Medium | 393.27 |
| 13.1627 | Medium | 646.86 |
| 15.953 | Medium | 771.5 |
| 16.5151 | Medium | 583.33 |
| 18.3244 | Low | 173.45 |
| 19.6593 | Low | 148.27 |
| 20.5291 | Low | 159.76 |
| 22.0131 | Low | 182.39 |
| 24.1905 | Low | 230.06 |
| 24.9829 | Low | 214.4 |
| 27.2598 | Low | 133.48 |

### Example 5: Characterization of Glutamate Salt Form A of Compound 1

As shown in Table 1, glutamate salt Form A of Compound 1 was obtained with the slurry methods as described in Example 2 from a mixture of the free base of Compound 1, glutamic acid, and acetone/H₂O (1:1, v/v). The solids were characterized by DSC/TGA and XRPD, as shown in FIGS. 3A and 3B, respectively. Table 5 summarizes the characterization of glutamate salt Form A of Compound 1.

TABLE 5

| | |
|---|---|
| Sample | Glutamate Salt Form A |
| Crystallinity by XRPD | High |
| Wt. Loss in TGA (%, ° C.) | 6.50 (150) |
| Endotherm in DSC (peak, ° C.) | 50.93 |
| | 94.60 |
| | 174.41 |
| Form | Hydrate/solvate |

Table 6 describes the XRD pattern of glutamate salt Form A crystalline form of Compound 1 shown in FIG. 3B.

TABLE 6

| Pos. (°2θ) | Intensity | Height (cts) |
|---|---|---|
| 5.1591 | Low | 318.71 |
| 9.9101 | High | 1366.22 |
| 10.2761 | Medium | 534.3 |
| 11.5649 | Low | 146.54 |
| 13.3175 | Medium | 580.03 |
| 14.348 | Medium | 749.15 |
| 15.4065 | Medium | 487.98 |
| 16.8829 | Medium | 839.65 |
| 17.635 | Medium | 440.38 |
| 18.5791 | Low | 282.32 |
| 19.8852 | Low | 227.79 |
| 20.5545 | Medium | 453.04 |
| 21.3977 | Low | 226.03 |
| 22.4135 | High | 1428.45 |
| 23.0565 | Medium | 439.1 |
| 24.5885 | High | 1692.37 |
| 25.2268 | High | 2369.01 |
| 26.0554 | Low | 190.61 |
| 27.3955 | Low | 240.62 |
| 28.1213 | Low | 189.13 |
| 28.7498 | Low | 319.08 |
| 31.0314 | Low | 244.11 |
| 32.7424 | Low | 198 |
| 34.7035 | Low | 147.81 |
| 37.4666 | Low | 133.56 |

### Example 6: Characterization of Fumarate Salt Form A and Form B of Compound 1

As shown in Table 1, fumarate salt Form A and Form B of Compound 1 was obtained with the slurry methods as described in Example 2. Fumarate salt Form A was obtained from a mixture of the free base of Compound 1, fumaric acid, and THF, while fumarate salt Form B was obtained from a mixture of the free base of Compound 1, fumaric acid, and acetone/H₂O (1:1, v/v). The solids were characterized by DSC/TGA and XRPD, as shown in FIGS. 4A, 4B, 4C, and 4D. Table 7 summarizes the characterization of fumarate salt Form A and Form B of Compound 1.

TABLE 7

| | | |
|---|---|---|
| Sample | Fumarate Salt Form A | Fumarate Salt Form B |
| Crystallinity by XRPD | Low | Medium |
| Wt. Loss in TGA | 7.02 (200) | 3.87 (220) |
| (%, ° C.) | | |
| Endotherm in DSC | 35.96 | 259.01 |
| (peak, ° C.) | 256.14 | |
| Form | Hydrate/solvate | Hydrate/solvate |

Table 8 describes the XRD pattern of fumarate salt Form A crystalline form of Compound 1 shown in FIG. 4B.

TABLE 8

| Pos. (°2θ) | Intensity | Height (cts) |
|---|---|---|
| 4.2915 | Medium | 476.8 |
| 6.0138 | Low | 107.04 |
| 8.0589 | Low | 80.79 |
| 9.7767 | Low | 118.24 |
| 15.5025 | Low | 74.19 |
| 16.1608 | Low | 67.4 |
| 19.6305 | Low | 90.13 |
| 21.2836 | Low | 54.9 |
| 25.0472 | Low | 95.46 |

Table 9 describes the XRD pattern of fumarate salt Form B crystalline form of Compound 1 shown in FIG. 4D.

TABLE 9

| Pos. (°2θ) | Intensity | Height (cts) |
|---|---|---|
| 3.6566 | Medium | 665.43 |
| 11.2791 | Low | 107.9 |
| 14.3561 | Low | 244.11 |
| 16.9732 | Low | 101.7 |
| 17.7986 | Low | 154.07 |
| 18.283 | Low | 115.26 |
| 20.6372 | Low | 243.45 |
| 22.2176 | Low | 104.41 |
| 23.706 | Low | 286.88 |
| 25.3126 | Medium | 494.6 |
| 26.958 | Low | 271.59 |
| 33.2918 | Low | 33.13 |

### Example 7: Characterization of Glycolate Salt Form A and Form B of Compound 1

As shown in Table 1, glycolate salt Form A and Form B of Compound 1 was obtained with the slurry methods as described in Example 2. Glycolate salt Form A was obtained from a mixture of the free base of Compound 1, glycolic acid, and methanol, while glycolate salt Form B was obtained from a mixture of the free base of Compound 1, glycolic acid, and THF. The solids were characterized by DSC/TGA and XRPD, as shown in FIGS. 5A, 5B, 5C, and 5D. Table 10 summarizes the characterization of glycolate salt Form A and Form B of Compound 1.

TABLE 10

| | | |
|---|---|---|
| Sample | Glycolate Salt Form A | Glycolate Salt Form B |
| Crystallinity by XRPD | Medium | Low |
| Wt. Loss in TGA | 2.08 (225) | 1.34 (100) |
| (%, ° C.) | | |
| Endotherm in DSC | 35.70 | 55.75 |
| (peak, ° C.) | 253.55 | 138.28 |
| Exotherm in DSC | 119.25 | — |
| (peak, ° C.) | 160.76 | |
| Form | Hydrate/solvate | Hydrate/solvate |
| | | or anhydrate |

Table 11 describes the XRD pattern of glycolate salt Form A crystalline form of Compound 1 shown in FIG. 5B.

TABLE 11

| Pos. (°2θ) | Intensity | Height (cts) |
|---|---|---|
| 8.0314 | Low | 220.35 |
| 9.7723 | Medium | 340.32 |
| 11.1646 | Low | 140.23 |
| 13.9563 | Low | 41.13 |
| 15.3032 | Low | 264.96 |
| 16.099 | Low | 255.25 |
| 19.4023 | Low | 187.96 |
| 19.879 | Low | 184.97 |
| 22.4702 | Low | 140.94 |
| 23.7495 | Low | 180.95 |
| 24.9932 | Medium | 360.94 |
| 25.5169 | Low | 137 |
| 26.432 | Low | 64.37 |
| 28.0825 | Low | 84.21 |

Table 12 describes the XRD pattern of glycolate salt Form B crystalline form of Compound 1 shown in FIG. 5D.

TABLE 12

| Pos. (°2θ) | Intensity | Height (cts) |
|---|---|---|
| 2.9632 | Medium | 586.52 |
| 5.781 | High | 1966.82 |
| 7.1493 | Low | 313.62 |
| 8.3987 | Medium | 584.06 |
| 10.189 | Medium | 447.41 |
| 12.2186 | Low | 147.8 |
| 13.9244 | Low | 195.21 |
| 15.4097 | Low | 109.24 |
| 16.3108 | Low | 160.32 |
| 17.0121 | Low | 160.71 |
| 26.9895 | Medium | 361.49 |

### Example 8: Characterization of Hippurate Salt Form A of Compound 1

As shown in Table 1, hippurate salt Form A of Compound 1 was obtained with the slurry methods as described in Example 2 from a mixture of the free base of Compound 1, hippuric acid, and THF. The solids were characterized by DSC/TGA and XRPD, as shown in FIGS. 6A and 6B. FIG. 6C shows a polarized light microscopy (PLM) image of the solids obtained. Table 13 summarizes the characterization of hippurate salt Form A of Compound 1.

TABLE 13

| | |
|---|---|
| Sample | Hippurate Salt Form A |
| Crystallinity by XRPD | High |
| Wt. Loss in TGA (%, ° C.) | 2.72 (140) |
| Endotherm in DSC (peak, ° C.) | 35.56 |
| | 177.67 |
| | 189.88 |
| Form | Hydrate/solvate or anhydrate |

Table 14 describes the XRD pattern of hippurate salt Form A crystalline form of Compound 1 shown in FIG. 6B.

TABLE 14

| Pos. (°2θ) | Intensity | Height (cts) |
|---|---|---|
| 3.2397 | High | 1118.76 |
| 9.4763 | Low | 240.16 |
| 9.7786 | Low | 150.72 |
| 11.0376 | Medium | 375.74 |
| 12.017 | Medium | 466.9 |
| 12.6331 | Low | 289.36 |
| 13.9211 | Low | 318.3 |
| 15.016 | Low | 151.91 |
| 15.7611 | Medium | 579.6 |
| 17.5346 | Low | 301.26 |
| 18.562 | Low | 249.9 |
| 19.0649 | Medium | 809.12 |
| 20.8452 | High | 1148.47 |
| 22.2098 | Medium | 458.77 |
| 22.7504 | Low | 302.37 |
| 23.4318 | Low | 300.06 |
| 24.945 | Medium | 398.69 |
| 26.0689 | Low | 192.22 |
| 26.8308 | Low | 292.68 |
| 27.4158 | High | 919.77 |
| 28.0299 | Medium | 484.23 |
| 28.9095 | Low | 332.02 |
| 30.0777 | Low | 144.69 |

### Example 9: Characterization of Phosphate Salt Form A of Compound 1

As shown in Table 1, phosphate salt Form A of Compound 1 was obtained with slurry methods as Example 2 from a mixture of the free base of Compound 1, phosphoric acid, and methanol. The solids were characterized by DSC/TGA and XRPD, as shown in FIGS. 7A and 7B, respectively. Table 15 summarizes the characterization of phosphate salt Form A of Compound 1.

TABLE 15

| | |
|---|---|
| Sample | Phosphate Salt Form A |
| Crystallinity by XRPD | High |
| Wt. Loss in TGA (%, ° C.) | 2.17 |
| Endotherm in DSC (peak, ° C.) | 35.81 |
| | 81.65 |
| | 161.86 |
| Form | Hydrate/solvate |

Table 16 describes the XRD pattern of phosphate salt Form A crystalline form of Compound 1 shown in FIG. 7B.

TABLE 16

| Pos. (°2θ) | Intensity | Height (cts) |
|---|---|---|
| 6.6113 | High | 1001.96 |
| 10.9662 | Medium | 339.58 |
| 11.6831 | Low | 81.7 |
| 12.879 | Low | 113.03 |
| 14.791 | Low | 110.71 |
| 16.4822 | Low | 279.99 |
| 17.0296 | Medium | 354.94 |
| 17.9287 | Low | 178.07 |
| 18.8999 | Medium | 393.81 |
| 19.8809 | Low | 317.09 |
| 20.3998 | Medium | 570.12 |
| 22.9003 | High | 2588.56 |
| 24.7426 | Low | 210.43 |
| 26.0253 | Low | 168.45 |
| 26.5477 | Low | 143.82 |
| 28.5068 | Low | 230.27 |
| 29.2913 | Low | 305.63 |
| 31.4136 | Low | 70.04 |

### Itemized List of Embodiments

Embodiment 1. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile sulfate having an X-ray powder diffraction (XRPD) pattern comprising a peak at a two-theta angle of 6.6°±0.2.

Embodiment 2. The crystalline form of embodiment 1, wherein the crystalline form has an X-ray powder diffraction (XRPD) pattern comprising a peak at a two-theta angle of 10.9°±0.2°.

Embodiment 3. The crystalline form of embodiment 1, wherein the crystalline form has an X-ray powder diffraction (XRPD) pattern comprising a peak at a two-theta angle of 20.4°±0.2°.

Embodiment 4. The crystalline form of embodiment 1, wherein the crystalline form has an X-ray powder diffraction (XRPD) pattern comprising a peak at a two-theta angle of 22.9°±0.2°

Embodiment 5. The crystalline form of embodiment 1, wherein the crystalline form has an X-ray powder diffraction (XRPD) pattern comprising peaks at a two-theta angle of 6.6°±0.2°, 10.9°±0.2°, 20.4°±0.2°, and 22.9°±0.2°.

Embodiment 6. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile sulfate having an X-ray powder diffraction pattern as shown in FIG. 1A.

Embodiment 7. The crystalline form of any of embodiments 1-3, wherein the crystalline form has a purity of at least 97% by weight of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile sulfate.

Embodiment 8. The crystalline form of any of embodiments 1-7, wherein the crystalline form has a purity of at least 98% by weight of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile sulfate.

Embodiment 9. A pharmaceutical composition comprising the crystalline form of any of embodiments 1-8 and a pharmaceutically acceptable carrier.

Embodiment 10. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile tosylate having an endothermic differential scanning calorimetric (DSC) peak temperature within ±2% of 55° C.

Embodiment 11. The crystalline form of embodiment 10, wherein the endothermic DSC peak temperature is within ±1% of 55° C.

Embodiment 12. The crystalline form of embodiment 10, wherein the endothermic DSC peak temperature is within ±0.5% of 55° C.

Embodiment 13. The crystalline form of embodiment 10, wherein the crystalline form has an endothermic DSC peak temperature within ±2% of 163° C.

Embodiment 14. The crystalline form of embodiment 13, wherein the endothermic DSC peak temperature is within ±1% of 163° C.

Embodiment 15. The crystalline form of embodiment 13, wherein the endothermic DSC peak temperature is within =0.5% of 163° C.

Embodiment 16. The crystalline form of embodiment 10, wherein the crystalline form has an exothermic DSC peak temperature within ±2% of 247° C.

Embodiment 17. The crystalline form of embodiment 16, wherein the exothermic DSC peak temperature is within ±1% of 247° C.

Embodiment 18. The crystalline form of embodiment 16, wherein the exothermic DSC peak temperature is within ±0.5% of 247° C.

Embodiment 19. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile tosylate having an X-ray powder diffraction (XRPD) pattern comprising a peak at a two-theta angle of 4.4°±0.2°.

Embodiment 20. The crystalline form of embodiment 19, wherein the crystalline form has an X-ray powder diffraction (XRPD) pattern comprising a peak at a two-theta angle of 5.2°±0.2°.

Embodiment 21. The crystalline form of embodiment 19, wherein the crystalline form has an X-ray powder diffraction (XRPD) pattern comprising a peak at a two-theta angle of 8.2°±0.2°.

Embodiment 22. The crystalline form of embodiment 19, wherein the crystalline form has an X-ray powder diffraction (XRPD) pattern comprising a peak at a two-theta angle of 13.1°±0.2°.

Embodiment 23. The crystalline form of embodiment 19, wherein the crystalline form has an X-ray powder diffraction (XRPD) pattern comprising a peak at a two-theta angle of 15.9°±0.2°.

Embodiment 24. The crystalline form of embodiment 19, wherein the crystalline form has an X-ray powder diffraction (XRPD) pattern comprising a peak at a two-theta angle of 16.5°±0.2°.

Embodiment 25. The crystalline form of embodiment 19, wherein the crystalline form has an X-ray powder diffraction (XRPD) pattern comprising peaks at a two-theta angle of 4.4°±0.2°, 5.2°±0.2°, 8.2°±0.2°, 13.1°±0.2°, 15.9°±0.2°, and 16.5°±0.2°.

Embodiment 26. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile tosylate having an X-ray powder diffraction pattern as shown in FIG. 2B.

Embodiment 27. The crystalline form of any of embodiments 10-26, wherein the crystalline form is a hydrate, solvate, or an anhydrate.

Embodiment 28. The crystalline form of any of embodiments 10-27, wherein the crystalline form has a thermal gravimetric analysis (TGA) plot comprising a mass loss of about 2.7% when heated from about 25° C. to about 150° C.

Embodiment 29. The crystalline form of any of embodiments 10-28, wherein the crystalline form has a purity of at least 97% by weight of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile tosylate.

Embodiment 30. The crystalline form of any of embodiments 10-29, wherein the crystalline form has a purity of at least 98% by weight of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile tosylate.

Embodiment 31. A pharmaceutical composition comprising the crystalline form of any of embodiments 10-30 and a pharmaceutically acceptable carrier.

Embodiment 32. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile glutamate having an endothermic differential scanning calorimetric (DSC) peak temperature within ±2% of 51° C.

Embodiment 33. The crystalline form of embodiment 32, wherein the endothermic DSC peak temperature is within ±1% of 51° C.

Embodiment 34. The crystalline form of embodiment 32, wherein the endothermic DSC peak temperature is within ±0.5% of 51° C.

Embodiment 35. The crystalline form of embodiment 32, wherein the crystalline form has an endothermic DSC peak temperature within ±2% of 94° C.

Embodiment 36. The crystalline form of embodiment 25, wherein the endothermic DSC peak temperature is within ±1% of 94° C.

Embodiment 37. The crystalline form of embodiment 25, wherein the endothermic DSC peak temperature is within ±0.5% of 94° C.

Embodiment 38. The crystalline form of embodiment 32, wherein the crystalline form has an endothermic DSC peak temperature within ±2% of 174° C.

Embodiment 39. The crystalline form of embodiment 38, wherein the endothermic DSC peak temperature is within ±1% of 174° C.

Embodiment 40. The crystalline form of embodiment 38, wherein the endothermic DSC peak temperature is within ±0.5% of 174° C.

Embodiment 41. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile glutamate having an X-ray powder diffraction (XRPD) pattern comprising a peak at a two-theta angle of 9.9°±0.2°.

Embodiment 42. The crystalline form of embodiment 41, wherein the crystalline form has an X-ray powder diffraction (XRPD) pattern comprising a peak at a two-theta angle of 14.3°±0.2°.

Embodiment 43. The crystalline form of embodiment 41, wherein the crystalline form has an X-ray powder diffraction (XRPD) pattern comprising a peak at a two-theta angle of 16.9°±0.2°.

Embodiment 44. The crystalline form of embodiment 41, wherein the crystalline form has an X-ray powder diffraction (XRPD) pattern comprising a peak at a two-theta angle of 22.4°±0.2°.

Embodiment 45. The crystalline form of embodiment 41, wherein the crystalline form has an X-ray powder diffraction (XRPD) pattern comprising a peak at a two-theta angle of 24.6°±0.2°.

Embodiment 46. The crystalline form of embodiment 41, wherein the crystalline form has an X-ray powder diffraction (XRPD) pattern comprising a peak at a two-theta angle of 25.2°±0.2°.

Embodiment 47. The crystalline form of embodiment 41, wherein the crystalline form has an X-ray powder diffraction (XRPD) pattern comprising peaks at a two-theta angle 9.9°±0.2°, 14.3°±0.2°, 16.9°±0.2°, 22.4°±0.2°, 24.6°±0.2°, and 25.2°±0.2°.

Embodiment 48. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile glutamate having an X-ray powder diffraction pattern as shown in FIG. 3B.

Embodiment 49. The crystalline form of any of embodiments 32-48, wherein the crystalline form is a hydrate or solvate.

Embodiment 50. The crystalline form of any of embodiments 32-49, wherein the crystalline form has a thermal gravimetric analysis (TGA) plot comprising a mass loss of about 6.5% when heated from about 25° C. to about 150° C.

Embodiment 51. The crystalline form of any of embodiments 32-50, wherein the crystalline form has a purity of at least 97% by weight of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile glutamate.

Embodiment 52. The crystalline form of any of embodiments 32-51, wherein the crystalline form has a purity of at least 98% by weight of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile glutamate.

Embodiment 53. A pharmaceutical composition comprising the crystalline form of any of embodiments 32-52 and a pharmaceutically acceptable carrier.

Embodiment 54. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile fumarate having an endothermic differential scanning calorimetric (DSC) peak temperature within ±2% of 36° C.

Embodiment 55. The crystalline form of embodiment 54, wherein the endothermic DSC peak temperature is within ±1% of 36° C.

Embodiment 56. The crystalline form of embodiment 54, wherein the endothermic DSC peak temperature is within ±0.5% of 36° C.

Embodiment 57. The crystalline form of embodiment 54, wherein the crystalline form has an endothermic DSC peak temperature within ±2% of 256° C.

Embodiment 58. The crystalline form of embodiment 57, wherein the endothermic DSC peak temperature is within ±1% of 256° C.

Embodiment 59. The crystalline form of embodiment 57, wherein the endothermic DSC peak temperature is within ±0.5% of 256° C.

Embodiment 60. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile fumarate having an X-ray powder diffraction (XRPD) pattern comprising a peak at a two-theta angle of 4.3°±0.2°.

Embodiment 61. The crystalline form of embodiment 60, wherein the crystalline form has an X-ray powder diffraction (XRPD) pattern comprising a peak at a two-theta angle of 6.0°±0.2°.

Embodiment 62. The crystalline form of embodiment 60, wherein the crystalline form has an X-ray powder diffraction (XRPD) pattern comprising a peak at a two-theta angle of 9.8°±0.2°.

Embodiment 63. The crystalline form of embodiment 60, wherein the crystalline form has an X-ray powder diffraction (XRPD) pattern comprising a peak at a two-theta angle of 19.6°±0.2°.

Embodiment 64. The crystalline form of embodiment 60, wherein the crystalline form has an X-ray powder diffraction (XRPD) pattern comprising a peak at a two-theta angle of 25.0°±0.2°.

Embodiment 65. The crystalline form of embodiment 60, wherein the crystalline form has an X-ray powder diffraction (XRPD) pattern comprising peaks at a two-theta angle of 4.3°±0.2°, 6.0°±0.2°, 9.8°±0.2°, 19.6°±0.2°, and 25.0°±0.2°

Embodiment 66. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile fumarate having an X-ray powder diffraction pattern as shown in FIG. 4B.

Embodiment 67. The crystalline form of any of embodiments 54-66, wherein the crystalline form is a hydrate or solvate.

Embodiment 68. The crystalline form of any of embodiments 54-67, wherein the crystalline form has a thermal gravimetric analysis (TGA) plot comprising a mass loss of about 7.0% when heated from about 25° C. to about 150° C.

Embodiment 69. The crystalline form of any of embodiments 54-68, wherein the crystalline form has a purity of at least 97% by weight of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile fumarate.

Embodiment 70. The crystalline form of any of embodiments 54-69, wherein the crystalline form has a purity of at least 98% by weight of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile fumarate.

Embodiment 71. A pharmaceutical composition comprising the crystalline form of any of embodiments 54-70 and a pharmaceutically acceptable carrier.

Embodiment 72. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3 -fluorobenzonitrile fumarate having an endothermic differential scanning calorimetric (DSC) peak temperature within ±2% of 259° C.

Embodiment 73. The crystalline form of embodiment 72, wherein the endothermic DSC peak temperature is within ±1% of 259° C.

Embodiment 74. The crystalline form of embodiment 72, wherein the endothermic DSC peak temperature is within ±0.5% of 259° C.

Embodiment 75. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile fumarate having an X-ray powder diffraction (XRPD) pattern comprising a peak at a two-theta angle of 3.6°±0.2°.

Embodiment 76. The crystalline form of embodiment 75, wherein the crystalline form has an X-ray powder diffraction (XRPD) pattern comprising a peak at a two-theta angle of 14.3°±0.2°.

Embodiment 77. The crystalline form of embodiment 75, wherein the crystalline form has an X-ray powder diffraction (XRPD) pattern comprising a peak at a two-theta angle of 20.6°±0.2°.

Embodiment 78. The crystalline form of embodiment 75, wherein the crystalline form has an X-ray powder diffraction (XRPD) pattern comprising a peak at a two-theta angle of 23.7°±0.2°.

Embodiment 79. The crystalline form of embodiment 58, wherein the crystalline form has an X-ray powder diffraction (XRPD) pattern comprising a peak at a two-theta angle of 25.3°±0.2°.

Embodiment 80. The crystalline form of embodiment 75, wherein the crystalline form has an X-ray powder diffraction (XRPD) pattern comprising a peak at a two-theta angle of 26.9°±0.2°.

Embodiment 81. The crystalline form of embodiment 75, wherein the crystalline form has an X-ray powder diffraction (XRPD) pattern comprising peaks at a two-theta angle of 3.6°±0.2°, 14.3°±0.2°, 20.6°±0.2°, 23.7°±0.2°, 25.3°±0.2°, and 26.9°±0.2°.

Embodiment 82. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile fumarate having an X-ray powder diffraction pattern as shown in FIG. 4D.

Embodiment 83. The crystalline form of any of embodiments 72-82, wherein the crystalline form is a hydrate or solvate.

Embodiment 84. The crystalline for of any of embodiments 72-83, wherein the crystalline form has a thermal gravimetric analysis (TGA) plot comprising a mass low of about 3.9% when heated from about 25° C. to about 150° C.

Embodiment 85. The crystalline form of any of embodiments 72-84, wherein the crystalline form has a purity of at least 97% by weight of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile fumarate.

Embodiment 86. The crystalline form of any of embodiments 72-85, wherein the crystalline form has a purity of at least 98% by weight of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile fumarate.

Embodiment 87. A pharmaceutical composition comprising the crystalline form of any of embodiments 72-86 and a pharmaceutically acceptable carrier.

Embodiment 88. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile glycolate having an endothermic differential scanning calorimetric (DSC) peak temperature within ±2% of 36° C.

Embodiment 89. The crystalline form of embodiment 88, wherein the endothermic DSC peak temperature is within ±1% of 36° C.

Embodiment 90. The crystalline form of embodiment 88, wherein the endothermic DSC peak temperature is within ±0.5% of 36° C.

Embodiment 91. The crystalline form of embodiment 88, wherein the crystalline form has an exothermic DSC peak temperature within ±2% of 119° C.

Embodiment 92. The crystalline form of embodiment 91, wherein the exothermic DSC peak temperature is within ±1% of 119° C.

Embodiment 93. The crystalline form of embodiment 91, wherein the exothermic DSC peak temperature is within ±0.5% of 119° C.

Embodiment 94. The crystalline form of embodiment 88, wherein the crystalline form has an exothermic DSC peak temperature within ±2% of 161° C.

Embodiment 95. The crystalline form of embodiment 94, wherein the exothermic DSC peak temperature is within ±1% of 161° C.

Embodiment 96. The crystalline form of embodiment 94, wherein the exothermic DSC peak temperature is within ±0.5% of 161° C.

Embodiment 97. The crystalline form of embodiment 88, wherein the crystalline form has an endothermic DSC peak temperature within ±2% of 253° C.

Embodiment 98. The crystalline form of embodiment 97, wherein the endothermic DSC peak temperature is within ±1% of 253° C.

Embodiment 99. The crystalline form of embodiment 97, wherein the endothermic DSC peak temperature is within ±0.5% of 253° C.

Embodiment 100. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile glycolate having an X-ray powder diffraction (XRPD) pattern comprising a peak at a two-theta angle of 9.8°±0.2°.

Embodiment 101. The crystalline form of embodiment 100, wherein the crystalline form has an X-ray powder diffraction (XRPD) pattern comprising a peak at a two-theta angle of 15.3°±0.2°.

Embodiment 102. The crystalline form of embodiment 100, wherein the crystalline form has an X-ray powder diffraction (XRPD) pattern comprising a peak at a two-theta angle of 16.1°±0.2°.

Embodiment 103. The crystalline form of embodiment 100, wherein the crystalline form has an X-ray powder diffraction (XRPD) pattern comprising a peak at a two-theta angle of 25.0°±0.2°.

Embodiment 104. The crystalline form of embodiment 100, wherein the crystalline form has an X-ray powder diffraction (XRPD) pattern comprising peaks at a two-theta angle of 9.8°±0.2°, 15.3°±0.2°, 16.1°±0.2°, and 25.0°±0.2°.

Embodiment 105. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile glycolate having an X-ray powder diffraction pattern as shown in FIG. 5B.

Embodiment 106. The crystalline form of any of embodiments 88-105, wherein the crystalline form is a hydrate or solvate.

Embodiment 107. The crystalline form of any of embodiments 88-106, wherein the crystalline form has a thermal gravimetric analysis (TGA) plot comprising a mass loss of about 2.1% when heated from about 25° C. to about 150° C.

Embodiment 108. The crystalline form of any of embodiments 88-107, wherein the crystalline form has a purity of at least 97% by weight of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile glycolate.

Embodiment 109. The crystalline form of any of embodiments 88-108, wherein the crystalline form has a purity of at least 98% by weight of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile glycolate.

Embodiment 110. A pharmaceutical composition comprising the crystalline form of any of embodiments 88-109 and a pharmaceutically acceptable carrier.

Embodiment 111. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile glycolate having an endothermic differential scanning calorimetric (DSC) peak temperature within ±2% of 56° C.

Embodiment 112. The crystalline form of embodiment 111, wherein the endothermic DSC peak temperature is within ±1% of 56° C.

Embodiment 113. The crystalline form of embodiment 111, wherein the endothermic DSC peak temperature is within ±0.5% of 56° C.

Embodiment 114. The crystalline form of embodiment 111, wherein the crystalline form has an endothermic DSC peak temperature within ±2% of 138° C.

Embodiment 115. The crystalline form of embodiment 114, wherein the endothermic DSC peak temperature is within ±1% of 138° C.

Embodiment 116. The crystalline form of embodiment 114, wherein the endothermic DSC peak temperature is within ±0.5% of 138° C.

Embodiment 117. The crystalline form of embodiment 111, wherein the crystalline form has an exothermic DSC peak temperature within ±2% of 259° C.

Embodiment 118. The crystalline form of embodiment 117, wherein the exothermic DSC peak temperature is within ±1% of 259° C.

Embodiment 119. The crystalline form of embodiment 117, wherein the exothermic DSC peak temperature is within ±0.5% of 259° C.

Embodiment 120. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile glycolate having an X-ray powder diffraction (XRPD) pattern comprising a peak at a two-theta angle of 2.9°±0.2°.

Embodiment 121. The crystalline form of embodiment 120, wherein the crystalline form has an X-ray powder diffraction (XRPD) pattern comprising a peak at a two-theta angle of 5.8°±0.2°.

Embodiment 122. The crystalline form of embodiment 120, wherein the crystalline form has an X-ray powder diffraction (XRPD) pattern comprising a peak at a two-theta angle of 8.4°±0.2°.

Embodiment 123. The crystalline form of embodiment 120, wherein the crystalline form has an X-ray powder diffraction (XRPD) pattern comprising a peak at a two-theta angle of 10.2°±0.2°.

Embodiment 124. The crystalline form of embodiment 120, wherein the crystalline form has an X-ray powder diffraction (XRPD) pattern comprising a peak at a two-theta angle of 27.0°±0.2°

Embodiment 125. The crystalline form of embodiment 120, wherein the crystalline form has an X-ray powder diffraction (XRPD) pattern comprising peaks at a two-theta angle of 2.9°±0.2°, 5.8°±0.2°, 8.4°±0.2°, 10.2°±0.2°, and 27.0°±0.2°.

Embodiment 126. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile glycolate having an X-ray powder diffraction pattern as shown in FIG. 5D.

Embodiment 127. The crystalline form of any of embodiments 111-126, wherein the crystalline form is a hydrate, solvate, or an anhydrate.

Embodiment 128. The crystalline form of any of embodiments 111-127, wherein the crystalline form has a thermal gravimetric analysis (TGA) plot comprising a mass loss of about 1.3% when heated from about 25° C. to about 150° C.

Embodiment 129. The crystalline form of any of embodiments 111-128, wherein the crystalline form has a purity of at least 97% by weight of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile glycolate.

Embodiment 130. The crystalline form of any of embodiments 111-129, wherein the crystalline form has a purity of at least 98% by weight of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile glycolate.

Embodiment 131. A pharmaceutical composition comprising the crystalline form of any of embodiments 111-130 and a pharmaceutically acceptable carrier.

Embodiment 132. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3 -fluorobenzonitrile hippurate having an endothermic differential scanning calorimetric (DSC) peak temperature within ±2% of 36° C.

Embodiment 133. The crystalline form of embodiment 132, wherein the endothermic DSC peak temperature is within ±1% of 36° C.

Embodiment 134. The crystalline form of embodiment 132, wherein the endothermic DSC peak temperature is within ±0.5% of 36° C.

Embodiment 135. The crystalline form of embodiment 132, wherein the endothermic crystalline form has an DSC peak temperature within ±2% of 177° C.

Embodiment 136. The crystalline form of embodiment 135, wherein the endothermic DSC peak temperature is within ±1% of 177° C.

Embodiment 137. The crystalline form of embodiment 135, wherein the endothermic DSC peak temperature is within ±0.5% of 177° C.

Embodiment 138. The crystalline form of embodiment 132, wherein the crystalline form has an endothermic DSC peak temperature within ±2% of 208° C.

Embodiment 139. The crystalline form of embodiment 138, wherein the endothermic DSC peak temperature is within ±1% of 208° C.

Embodiment 140. The crystalline form of embodiment 138, wherein the endothermic DSC peak temperature is within ±0.5% of 208° C.

Embodiment 141. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile hippurate having an X-ray powder diffraction (XRPD) pattern comprising a peak at a two-theta angle of 3.2°±0.2°.

Embodiment 142. The crystalline form of embodiment 141, wherein the crystalline form has an X-ray powder diffraction (XRPD) pattern comprising a peak at a two-theta angle of 19.1°±0.2°.

Embodiment 143. The crystalline form of embodiment 141, wherein the crystalline form has an X-ray powder diffraction (XRPD) pattern comprising a peak at a two-theta angle of 20.8°±0.2°.

Embodiment 144. The crystalline form of embodiment 141, wherein the crystalline form has an X-ray powder diffraction (XRPD) pattern comprising a peak at a two-theta angle of 27.4°±0.2°.

Embodiment 145. The crystalline form of embodiment 141, wherein the crystalline form has an X-ray powder diffraction (XRPD) pattern comprising peaks at a two-theta angle of 3.2°±0.2°, 19.1°±0.2°, 20.8°±0.2°, and 27.4°±0.2°.

Embodiment 146. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile hippurate having an X-ray powder diffraction pattern as shown in FIG. 6B.

Embodiment 147. The crystalline form of any of embodiments 132-146, wherein the crystalline form is a hydrate, or solvate, or an anhydrate.

Embodiment 148. The crystalline form of any of embodiments 132-147, wherein the crystalline form has a thermal gravimetric analysis (TGA) plot comprising a mass loss of about 2.7% when heated from about 25° C. to about 150° C.

Embodiment 149. The crystalline form of any of embodiments 132-148, wherein the crystalline form has a purity of at least 97% by weight of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile hippurate.

Embodiment 150. The crystalline form of any of embodiments 132-149, wherein the crystalline form has a purity of at least 98% by weight of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile hippurate.

Embodiment 151. A pharmaceutical composition comprising the crystalline form of any of embodiments 132-150 and a pharmaceutically acceptable carrier.

Embodiment 152. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile phosphate having an endothermic differential scanning calorimetric (DSC) peak temperature within ±2% of 36° C.

Embodiment 153. The crystalline form of embodiment 152, wherein the endothermic DSC peak temperature is within ±1% of 36° C.

Embodiment 154. The crystalline form of embodiment 152, wherein the endothermic DSC peak temperature is within ±0.5% of 36° C.

Embodiment 155. The crystalline form of embodiment 152, wherein the endothermic crystalline form has an DSC peak temperature within ±2% of 82° C.

Embodiment 156. The crystalline form of embodiment 155, wherein the endothermic DSC peak temperature is within ±1% of 82° C.

Embodiment 157. The crystalline form of embodiment 155, wherein the endothermic DSC peak temperature is within ±0.5% of 82° C.

Embodiment 158. The crystalline form of embodiment 152, wherein the endothermic crystalline form has an DSC peak temperature within ±2% of 162° C.

Embodiment 159. The crystalline form of embodiment 158, wherein the endothermic DSC peak temperature is within ±1% of 162° C.

Embodiment 160. The crystalline form of embodiment 158, wherein the endothermic DSC peak temperature is within ±0.5% of 162° C.

Embodiment 161. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile phosphate having an X-ray powder diffraction (XRPD) pattern comprising a peak at a two-theta angle of 6.6°±0.2°.

Embodiment 162. The crystalline form of embodiment 161, wherein the crystalline form has an X-ray powder diffraction (XRPD) pattern comprising a peak at a two-theta angle of 17.0°±0.2°.

Embodiment 163. The crystalline form of embodiment 161, wherein the crystalline form has an X-ray powder diffraction (XRPD) pattern comprising a peak at a two-theta angle of 18.9°±0.2°.

Embodiment 164. The crystalline form of embodiment 161, wherein the crystalline form has an X-ray powder diffraction (XRPD) pattern comprising a peak at a two-theta angle of 20.4°±0.2°.

Embodiment 165. The crystalline form of embodiment 161, wherein the crystalline form has an X-ray powder diffraction (XRPD) pattern comprising a peak at a two-theta angle of 22.9°±0.2°

Embodiment 166. The crystalline form of embodiment 161, wherein the crystalline form has an X-ray powder diffraction (XRPD) pattern comprising peaks at a two-theta angle of 6.6°±0.2°, 17.0°±0.2°, 18.9°±0.2°, 20.4°±0.2°, and 22.9°±0.2°.

Embodiment 167. A crystalline form of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile phosphate having an X-ray powder diffraction pattern as shown in FIG. 7B.

Embodiment 168. The crystalline form of any of embodiments 152-167, wherein the crystalline form is a hydrate or solvate.

Embodiment 169. The crystalline form of any of embodiments 152-168, wherein the crystalline form has a thermal gravimetric analysis (TGA) plot comprising a mass loss of about 2.2% when heated from about 25° C. to about 150° C.

Embodiment 170. The crystalline form of any of embodiments 152-169, wherein the crystalline form has a purity of at least 97% by weight of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile phosphate.

Embodiment 171. The crystalline form of any of embodiments 152-170, wherein the crystalline form has a purity of at least 98% by weight of 2-(4-(4-(aminomethyl)-1-oxo-1,2-dihydrophthalazin-6-yl)-1-methyl-1 h-pyrazol-5-yl)-4-chloro-6-cyclopropoxy-3-fluorobenzonitrile phosphate.

Embodiment 172. A pharmaceutical composition comprising the crystalline form of any of embodiments 152-171 and a pharmaceutically acceptable carrier.

Embodiment 173. A method for treating cancer in a subject in need thereof, comprising administering to the subject a crystalline form of a crystalline form of any one of embodiments 1-8, 10-30, 32-52, 54-70, 72-86, 88-109, 111-130, 132-150 or 152-171, or a pharmaceutical composition comprising a crystalline form of any one of embodiments 9, 31, 53, 71, 87, 110, 131, 151 or 172.

Embodiment 174. A method according to Embodiment 173 wherein the cancer is a MTAP-associated cancer.

Embodiment 175. A method according to Embodiment 173 wherein the cancer is selected from the group consisting of ovarian serous cystadenocarcinoma, squamous cell lung cancer, lung adenocarcinoma, mesothelioma; esophageal squamous cell carcinoma, gastric adenocarcinoma, pancreatic ductal adenocarcinoma, kidney adenocarcinoma, bladder transitional cell carcinoma, hepatocellular carcinoma, cholangiocarcinoma, cholangiocarcinoma; osteosarcoma, multiple myeloma, astrocytoma, glioma, glioblastoma, uterine sarcoma, acute myeloid leukemia, acute lymphoblastic leukemia, non-Hodgkin's lymphoma, malignant melanoma, endometrial carcinoma and thyroid carcinoma.

Embodiment 176. A method according to Embodiment 173 wherein the cancer is selected from the group consisting of bladder cancer, bone cancer, brain cancer, blood cancer, breast cancer, colorectal cancer, esophageal cancer, gastric cancer, head and neck cancer, kidney cancer, liver cancer, lung cancer, mesothelioma, ovarian cancer, pancreatic cancer, skin cancer, thyroid cancer and uterine cancer.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

We claim:

1. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile sulfate having an X-ray powder diffraction (XRPD) pattern comprising a peak at a two-theta angle of 6.6°±0.2.

2. The crystalline form of claim 1, wherein the crystalline form has an X-ray powder diffraction (XRPD) pattern comprising a peak at a two-theta angle of 10.9°±0.2°.

3. The crystalline form of claim 1, wherein the crystalline form has an X-ray powder diffraction (XRPD) pattern comprising a peak at a two-theta angle of 20.4°±0.2°.

4. The crystalline form of claim 1, wherein the crystalline form has an X-ray powder diffraction (XRPD) pattern comprising a peak at a two-theta angle of 22.9°±0.2°.

5. The crystalline form of claim 1, wherein the crystalline form has an X-ray powder diffraction (XRPD) pattern comprising peaks at a two-theta angle of 6.6°±0.2°, 10.9°±0.2°, 20.4°±0.2°, and 22.9°±0.2°.

6. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile sulfate having an X-ray powder diffraction pattern as shown in FIG. 1A.

7. The crystalline form of any of claims 1-3, wherein the crystalline form has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile sulfate.

8. The crystalline form of any of claims 1-7, wherein the crystalline form has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile sulfate.

9. A pharmaceutical composition comprising the crystalline form of any of claims 1-8 and a pharmaceutically acceptable carrier.

10. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile tosylate having an endothermic differential scanning calorimetric (DSC) peak temperature within ±2% of 55° C.

11. The crystalline form of claim 10, wherein the endothermic DSC peak temperature is within ±1% of 55° C.

12. The crystalline form of claim 10, wherein the endothermic DSC peak temperature is within ±0.5% of 55° C.

13. The crystalline form of claim 10, wherein the crystalline form has an endothermic DSC peak temperature within ±2% of 163° C.

14. The crystalline form of claim 13, wherein the endothermic DSC peak temperature is within ±1% of 163° C.

15. The crystalline form of claim 13, wherein the endothermic DSC peak temperature is within ±0.5% of 163° C.

16. The crystalline form of claim 10, wherein the crystalline form has an exothermic DSC peak temperature within ±2% of 247° C.

17. The crystalline form of claim 16, wherein the exothermic DSC peak temperature is within ±1% of 247° C.

18. The crystalline form of claim 16, wherein the exothermic DSC peak temperature is within ±0.5% of 247° C.

19. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile tosylate having an X-ray powder diffraction (XRPD) pattern comprising a peak at a two-theta angle of 4.4°±0.2°.

20. The crystalline form of claim 19, wherein the crystalline form has an X-ray powder diffraction (XRPD) pattern comprising a peak at a two-theta angle of 5.2°±0.2°.

21. The crystalline form of claim 19, wherein the crystalline form has an X-ray powder diffraction (XRPD) pattern comprising a peak at a two-theta angle of 8.2°±0.2°.

22. The crystalline form of claim 19, wherein the crystalline form has an X-ray powder diffraction (XRPD) pattern comprising a peak at a two-theta angle of 13.1°±0.2°.

23. The crystalline form of claim 19, wherein the crystalline form has an X-ray powder diffraction (XRPD) pattern comprising a peak at a two-theta angle of 15.9°±0.2°.

24. The crystalline form of claim 19, wherein the crystalline form has an X-ray powder diffraction (XRPD) pattern comprising a peak at a two-theta angle of 16.5°±0.2°.

25. The crystalline form of claim 19, wherein the crystalline form has an X-ray powder diffraction (XRPD) pattern comprising peaks at a two-theta angle of 4.4°±0.2°, 5.2°±0.2°, 8.2°±0.2°, 13.1°±0.2°, 15.9°±0.2°, and 16.5°±0.2°.

26. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile tosylate having an X-ray powder diffraction pattern as shown in FIG. 2B.

27. The crystalline form of any of claims 10-26, wherein the crystalline form is a hydrate, solvate, or an anhydrate.

28. The crystalline form of any of claims 10-27, wherein the crystalline form has a thermal gravimetric analysis (TGA) plot comprising a mass loss of about 2.7% when heated from about 25° C. to about 150° C.

29. The crystalline form of any of claims 10-28, wherein the crystalline form has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile tosylate.

30. The crystalline form of any of claims 10-29, wherein the crystalline form has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile tosylate.

31. A pharmaceutical composition comprising the crystalline form of any of claims 10-30 and a pharmaceutically acceptable carrier.

32. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glutamate having an endothermic differential scanning calorimetric (DSC) peak temperature within ±2% of 51° C.

33. The crystalline form of claim 32, wherein the endothermic DSC peak temperature is within ±1% of 51° C.

34. The crystalline form of claim 32, wherein the endothermic DSC peak temperature is within ±0.5% of 51° C.

35. The crystalline form of claim 32, wherein the crystalline form has an endothermic DSC peak temperature within ±2% of 94° C.

36. The crystalline form of claim 25, wherein the endothermic DSC peak temperature is within ±1% of 94° C.

37. The crystalline form of claim 25, wherein the endothermic DSC peak temperature is within ±0.5% of 94° C.

38. The crystalline form of claim 32, wherein the crystalline form has an endothermic DSC peak temperature within ±2% of 174° C.

39. The crystalline form of claim 38, wherein the endothermic DSC peak temperature is within ±1% of 174° C.

40. The crystalline form of claim 38, wherein the endothermic DSC peak temperature is within ±0.5% of 174° C.

41. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glutamate having an X-ray powder diffraction (XRPD) pattern comprising a peak at a two-theta angle of 9.9°±0.2°.

42. The crystalline form of claim 41, wherein the crystalline form has an X-ray powder diffraction (XRPD) pattern comprising a peak at a two-theta angle of 14.3°±0.2°.

43. The crystalline form of claim 41, wherein the crystalline form has an X-ray powder diffraction (XRPD) pattern comprising a peak at a two-theta angle of 16.9°±0.2°.

44. The crystalline form of claim 41, wherein the crystalline form has an X-ray powder diffraction (XRPD) pattern comprising a peak at a two-theta angle of 22.4°±0.2°.

45. The crystalline form of claim 41, wherein the crystalline form has an X-ray powder diffraction (XRPD) pattern comprising a peak at a two-theta angle of 24.6°±0.2°.

46. The crystalline form of claim 41, wherein the crystalline form has an X-ray powder diffraction (XRPD) pattern comprising a peak at a two-theta angle of 25.2°±0.2°.

47. The crystalline form of claim 41, wherein the crystalline form has an X-ray powder diffraction (XRPD) pattern comprising peaks at a two-theta angle 9.9°±0.2°, 14.3°±0.2°, 16.9°±0.2°, 22.4°±0.2°, 24.6°±0.2°, and 25.2°±0.2°.

48. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glutamate having an X-ray powder diffraction pattern as shown in FIG. 3B.

49. The crystalline form of any of claims 32-48, wherein the crystalline form is a hydrate or solvate.

50. The crystalline form of any of claims 32-49, wherein the crystalline form has a thermal gravimetric analysis (TGA) plot comprising a mass loss of about 6.5% when heated from about 25° C. to about 150° C.

51. The crystalline form of any of claims 32-50, wherein the crystalline form has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glutamate.

52. The crystalline form of any of claims 32-51, wherein the crystalline form has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glutamate.

53. A pharmaceutical composition comprising the crystalline form of any of claims 32-52 and a pharmaceutically acceptable carrier.

54. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile fumarate having an endothermic differential scanning calorimetric (DSC) peak temperature within ±2% of 36° C.

55. The crystalline form of claim 54, wherein the endothermic DSC peak temperature is within ±1% of 36° C.

56. The crystalline form of claim 54, wherein the endothermic DSC peak temperature is within ±0.5% of 36° C.

57. The crystalline form of claim 54, wherein the crystalline form has an endothermic DSC peak temperature within ±2% of 256° C.

58. The crystalline form of claim 57, wherein the endothermic DSC peak temperature is within ±1% of 256° C.

59. The crystalline form of claim 57, wherein the endothermic DSC peak temperature is within ±0.5% of 256° C.

60. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile fumarate having an X-ray powder diffraction (XRPD) pattern comprising a peak at a two-theta angle of 4.3°±0.2°.

61. The crystalline form of claim 60, wherein the crystalline form has an X-ray powder diffraction (XRPD) pattern comprising a peak at a two-theta angle of 6.0°±0.2°.

62. The crystalline form of claim 60, wherein the crystalline form has an X-ray powder diffraction (XRPD) pattern comprising a peak at a two-theta angle of 9.8°±0.2°.

63. The crystalline form of claim 60, wherein the crystalline form has an X-ray powder diffraction (XRPD) pattern comprising a peak at a two-theta angle of 19.6°±0.2°.

64. The crystalline form of claim 60, wherein the crystalline form has an X-ray powder diffraction (XRPD) pattern comprising a peak at a two-theta angle of 25.0°±0.2°.

65. The crystalline form of claim 60, wherein the crystalline form has an X-ray powder diffraction (XRPD) pattern comprising peaks at a two-theta angle of 4.3°±0.2°, 6.0°±0.2°, 9.8°±0.2°, 19.6°±0.2°, and 25.0°±0.2°.

66. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile fumarate having an X-ray powder diffraction pattern as shown in FIG. 4B.

67. The crystalline form of any of claims 54-66, wherein the crystalline form is a hydrate or solvate.

68. The crystalline form of any of claims 54-67, wherein the crystalline form has a thermal gravimetric analysis (TGA) plot comprising a mass loss of about 7.0% when heated from about 25° C. to about 150° C.

69. The crystalline form of any of claims 54-68, wherein the crystalline form has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile fumarate.

70. The crystalline form of any of claims 54-69, wherein the crystalline form has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile fumarate.

71. A pharmaceutical composition comprising the crystalline form of any of claims 54-70 and a pharmaceutically acceptable carrier.

72. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile fumarate having an endothermic differential scanning calorimetric (DSC) peak temperature within ±2% of 259° C.

73. The crystalline form of claim 72, wherein the endothermic DSC peak temperature is within ±1% of 259° C.

74. The crystalline form of claim 72, wherein the endothermic DSC peak temperature is within ±0.5% of 259° C.

75. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile fumarate having an X-ray powder diffraction (XRPD) pattern comprising a peak at a two-theta angle of 3.6°±0.2°.

76. The crystalline form of claim 75, wherein the crystalline form has an X-ray powder diffraction (XRPD) pattern comprising a peak at a two-theta angle of 14.3°±0.2°.

77. The crystalline form of claim 75, wherein the crystalline form has an X-ray powder diffraction (XRPD) pattern comprising a peak at a two-theta angle of 20.6°±0.2°.

78. The crystalline form of claim 75, wherein the crystalline form has an X-ray powder diffraction (XRPD) pattern comprising a peak at a two-theta angle of 23.7°±0.2°.

79. The crystalline form of claim 58, wherein the crystalline form has an X-ray powder diffraction (XRPD) pattern comprising a peak at a two-theta angle of 25.3°±0.2°.

80. The crystalline form of claim 75, wherein the crystalline form has an X-ray powder diffraction (XRPD) pattern comprising a peak at a two-theta angle of 26.9°±0.2°.

81. The crystalline form of claim 75, wherein the crystalline form has an X-ray powder diffraction (XRPD) pattern comprising peaks at a two-theta angle of 3.6°±0.2°, 14.3°±0.2°, 20.6°±0.2°, 23.7°±0.2°, 25.3°±0.2°, and 26.9°±0.2°.

82. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile fumarate having an X-ray powder diffraction pattern as shown in FIG. 4D.

83. The crystalline form of any of claims 72-82, wherein the crystalline form is a hydrate or solvate.

84. The crystalline for of any of claims 72-83, wherein the crystalline form has a thermal gravimetric analysis (TGA) plot comprising a mass low of about 3.9% when heated from about 25° C. to about 150° C.

85. The crystalline form of any of claims 72-84, wherein the crystalline form has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile fumarate.

86. The crystalline form of any of claims 72-85, wherein the crystalline form has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile fumarate.

87. A pharmaceutical composition comprising the crystalline form of any of claims 72-86 and a pharmaceutically acceptable carrier.

88. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glycolate having an endothermic differential scanning calorimetric (DSC) peak temperature within ±2% of 36° C.

89. The crystalline form of claim 88, wherein the endothermic DSC peak temperature is within ±1% of 36° C.

90. The crystalline form of claim 88, wherein the endothermic DSC peak temperature is within ±0.5% of 36° C.

91. The crystalline form of claim 88, wherein the crystalline form has an exothermic DSC peak temperature within ±2% of 119° C.

92. The crystalline form of claim 91, wherein the exothermic DSC peak temperature is within ±1% of 119° C.

93. The crystalline form of claim 91, wherein the exothermic DSC peak temperature is within ±0.5% of 119° C.

94. The crystalline form of claim 88, wherein the crystalline form has an exothermic DSC peak temperature within ±2% of 161° C.

95. The crystalline form of claim 94, wherein the exothermic DSC peak temperature is within ±1% of 161° C.

96. The crystalline form of claim 94, wherein the exothermic DSC peak temperature is within ±0.5% of 161° C.

97. The crystalline form of claim 88, wherein the crystalline form has an endothermic DSC peak temperature within ±2% of 253° C.

98. The crystalline form of claim 97, wherein the endothermic DSC peak temperature is within ±1% of 253° C.

99. The crystalline form of claim 97, wherein the endothermic DSC peak temperature is within ±0.5% of 253° C.

100. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glycolate having an X-ray powder diffraction (XRPD) pattern comprising a peak at a two-theta angle of 9.8°±0.2°.

101. The crystalline form of claim 100, wherein the crystalline form has an X-ray powder diffraction (XRPD) pattern comprising a peak at a two-theta angle of 15.3°±0.2°.

102. The crystalline form of claim 100, wherein the crystalline form has an X-ray powder diffraction (XRPD) pattern comprising a peak at a two-theta angle of 16.1°±0.2°.

103. The crystalline form of claim 100, wherein the crystalline form has an X-ray powder diffraction (XRPD) pattern comprising a peak at a two-theta angle of 25.0°±0.2°.

104. The crystalline form of claim 100, wherein the crystalline form has an X-ray powder diffraction (XRPD) pattern comprising peaks at a two-theta angle of 9.8°±0.2°, 15.3°±0.2°, 16.1°±0.2°, and 25.0°±0.2°.

105. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glycolate having an X-ray powder diffraction pattern as shown in FIG. 5B.

106. The crystalline form of any of claims 88-105, wherein the crystalline form is a hydrate or solvate.

107. The crystalline form of any of claims 88-106, wherein the crystalline form has a thermal gravimetric analysis (TGA) plot comprising a mass loss of about 2.1% when heated from about 25° C. to about 150° C.

108. The crystalline form of any of claims 88-107, wherein the crystalline form has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glycolate.

109. The crystalline form of any of claims 88-108, wherein the crystalline form has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glycolate.

110. A pharmaceutical composition comprising the crystalline form of any of claims 88-109 and a pharmaceutically acceptable carrier.

111. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glycolate having an endothermic differential scanning calorimetric (DSC) peak temperature within ±2% of 56° C.

112. The crystalline form of claim 111, wherein the endothermic DSC peak temperature is within ±1% of 56° C.

113. The crystalline form of claim 111, wherein the endothermic DSC peak temperature is within ±0.5% of 56° C.

114. The crystalline form of claim 111, wherein the crystalline form has an endothermic DSC peak temperature within ±2% of 138° C.

115. The crystalline form of claim 114, wherein the endothermic DSC peak temperature is within ±1% of 138° C.

116. The crystalline form of claim 114, wherein the endothermic DSC peak temperature is within ±0.5% of 138° C.

117. The crystalline form of claim 111, wherein the crystalline form has an exothermic DSC peak temperature within ±2% of 259° C.

118. The crystalline form of claim 117, wherein the exothermic DSC peak temperature is within ±1% of 259° C.

119. The crystalline form of claim 117, wherein the exothermic DSC peak temperature is within ±0.5% of 259° C.

120. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glycolate having an X-ray powder diffraction (XRPD) pattern comprising a peak at a two-theta angle of 2.9°±0.2°.

121. The crystalline form of claim 120, wherein the crystalline form has an X-ray powder diffraction (XRPD) pattern comprising a peak at a two-theta angle of 5.8°±0.2°.

122. The crystalline form of claim 120, wherein the crystalline form has an X-ray powder diffraction (XRPD) pattern comprising a peak at a two-theta angle of 8.4°±0.2°.

123. The crystalline form of claim 120, wherein the crystalline form has an X-ray powder diffraction (XRPD) pattern comprising a peak at a two-theta angle of 10.2°±0.2°.

124. The crystalline form of claim 120, wherein the crystalline form has an X-ray powder diffraction (XRPD) pattern comprising a peak at a two-theta angle of 27.0°±0.2°.

125. The crystalline form of claim 120, wherein the crystalline form has an X-ray powder diffraction (XRPD) pattern comprising peaks at a two-theta angle of 2.9°±0.2°, 5.8°±0.2°, 8.4°±0.2°, 10.2°±0.2°, and 27.0°±0.2°.

126. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glycolate having an X-ray powder diffraction pattern as shown in FIG. 5D.

127. The crystalline form of any of claims 111-126, wherein the crystalline form is a hydrate, solvate, or an anhydrate.

128. The crystalline form of any of claims 111-127, wherein the crystalline form has a thermal gravimetric analysis (TGA) plot comprising a mass loss of about 1.3% when heated from about 25° C. to about 150° C.

129. The crystalline form of any of claims 111-128, wherein the crystalline form has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glycolate.

130. The crystalline form of any of claims 111-129, wherein the crystalline form has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile glycolate.

131. A pharmaceutical composition comprising the crystalline form of any of claims 111-130 and a pharmaceutically acceptable carrier.

132. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hippurate having an endothermic differential scanning calorimetric (DSC) peak temperature within ±2% of 36° C.

133. The crystalline form of claim 132, wherein the endothermic DSC peak temperature is within ±1% of 36° C.

134. The crystalline form of claim 132, wherein the endothermic DSC peak temperature is within ±0.5% of 36° C.

135. The crystalline form of claim 132, wherein the endothermic crystalline form has an DSC peak temperature within ±2% of 177° C.

136. The crystalline form of claim 135, wherein the endothermic DSC peak temperature is within ±1% of 177° C.

137. The crystalline form of claim 135, wherein the endothermic DSC peak temperature is within ±0.5% of 177° C.

138. The crystalline form of claim 132, wherein the crystalline form has an endothermic DSC peak temperature within ±2% of 208° C.

139. The crystalline form of claim 138, wherein the endothermic DSC peak temperature is within ±1% of 208° C.

140. The crystalline form of claim 138, wherein the endothermic DSC peak temperature is within =0.5% of 208° C.

141. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hippurate having an X-ray powder diffraction (XRPD) pattern comprising a peak at a two-theta angle of 3.2°±0.2°.

142. The crystalline form of claim 141, wherein the crystalline form has an X-ray powder diffraction (XRPD) pattern comprising a peak at a two-theta angle of 19.1°±0.2°.

143. The crystalline form of claim 141, wherein the crystalline form has an X-ray powder diffraction (XRPD) pattern comprising a peak at a two-theta angle of 20.8°±0.2°.

144. The crystalline form of claim 141, wherein the crystalline form has an X-ray powder diffraction (XRPD) pattern comprising a peak at a two-theta angle of 27.4°±0.2°.

145. The crystalline form of claim 141, wherein the crystalline form has an X-ray powder diffraction (XRPD) pattern comprising peaks at a two-theta angle of 3.2°±0.2°, 19.1°±0.2°, 20.8°±0.2°, and 27.4°±0.2°.

146. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hippurate having an X-ray powder diffraction pattern as shown in FIG. 6B.

147. The crystalline form of any of claims 132-146, wherein the crystalline form is a hydrate, or solvate, or an anhydrate.

148. The crystalline form of any of claims 132-147, wherein the crystalline form has a thermal gravimetric analysis (TGA) plot comprising a mass loss of about 2.7% when heated from about 25° C. to about 150° C.

149. The crystalline form of any of claims 132-148, wherein the crystalline form has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hippurate.

150. The crystalline form of any of claims 132-149, wherein the crystalline form has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hippurate.

151. A pharmaceutical composition comprising the crystalline form of any of claims 132-150 and a pharmaceutically acceptable carrier.

152. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile phosphate having an endothermic differential scanning calorimetric (DSC) peak temperature within ±2% of 36° C.

153. The crystalline form of claim 152, wherein the endothermic DSC peak temperature is within ±1% of 36° C.

154. The crystalline form of claim 152, wherein the endothermic DSC peak temperature is within ±0.5% of 36° C.

155. The crystalline form of claim 152, wherein the endothermic crystalline form has an DSC peak temperature within ±2% of 82° C.

156. The crystalline form of claim 155, wherein the endothermic DSC peak temperature is within ±1% of 82° C.

157. The crystalline form of claim 155, wherein the endothermic DSC peak temperature is within ±0.5% of 82° C.

158. The crystalline form of claim 152, wherein the endothermic crystalline form has an DSC peak temperature within ±2% of 162° C.

159. The crystalline form of claim 158, wherein the endothermic DSC peak temperature is within ±1% of 162° C.

160. The crystalline form of claim 158, wherein the endothermic DSC peak temperature is within ±0.5% of 162° C.

161. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile phosphate having an X-ray powder diffraction (XRPD) pattern comprising a peak at a two-theta angle of 6.6°±0.2°.

162. The crystalline form of claim 161, wherein the crystalline form has an X-ray powder diffraction (XRPD) pattern comprising a peak at a two-theta angle of 17.0°±0.2°.

163. The crystalline form of claim 161, wherein the crystalline form has an X-ray powder diffraction (XRPD) pattern comprising a peak at a two-theta angle of 18.9°±0.2°.

164. The crystalline form of claim 161, wherein the crystalline form has an X-ray powder diffraction (XRPD) pattern comprising a peak at a two-theta angle of 20.4°±0.2°.

165. The crystalline form of claim 161, wherein the crystalline form has an X-ray powder diffraction (XRPD) pattern comprising a peak at a two-theta angle of 22.9°±0.2°.

166. The crystalline form of claim 161, wherein the crystalline form has an X-ray powder diffraction (XRPD) pattern comprising peaks at a two-theta angle of 6.6°±0.2°, 17.0°±0.2°, 18.9°±0.2°, 20.4°±0.2°, and 22.9°±0.2°.

167. A crystalline form of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile phosphate having an X-ray powder diffraction pattern as shown in FIG. 7B.

168. The crystalline form of any of claims 152-167, wherein the crystalline form is a hydrate or solvate.

169. The crystalline form of any of claims 152-168, wherein the crystalline form has a thermal gravimetric analysis (TGA) plot comprising a mass loss of about 2.2% when heated from about 25° C. to about 150° C.

170. The crystalline form of any of claims 152-169, wherein the crystalline form has a purity of at least 97% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile phosphate.

171. The crystalline form of any of claims 152-170, wherein the crystalline form has a purity of at least 98% by weight of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile phosphate.

172. A pharmaceutical composition comprising the crystalline form of any of claims 152-171 and a pharmaceutically acceptable carrier.

173. A method for treating cancer in a subject in need thereof, comprising administering to the subject a crystalline form of a crystalline form of any one of claim 1-8, 10-30, 32-52, 54-70, 72-86, 88-109, 111-130, 132-150 or 152-171, or a pharmaceutical composition comprising a crystalline form of any one of claim 9, 31, 53, 71, 87, 110, 131, 151 or 172.

174. A method according to claim 173 wherein the cancer is a MTAP-associated cancer.

175. A method according to claim 173 wherein the cancer is selected from the group consisting of ovarian serous cystadenocarcinoma, squamous cell lung cancer, lung adenocarcinoma, mesothelioma; esophageal squamous cell carcinoma, gastric adenocarcinoma, pancreatic ductal adenocarcinoma, kidney adenocarcinoma, bladder transitional cell carcinoma, hepatocellular carcinoma, cholangiocarcinoma, cholangiocarcinoma; osteosarcoma, multiple myeloma, astrocytoma, glioma, glioblastoma, uterine sarcoma, acute myeloid leukemia, acute lymphoblastic leukemia, non-Hodgkin's lymphoma, malignant melanoma, endometrial carcinoma and thyroid carcinoma.

176. A method according to claim 173 wherein the cancer is selected from the group consisting of bladder cancer, bone cancer, brain cancer, blood cancer, breast cancer, colorectal cancer, esophageal cancer, gastric cancer, head and neck cancer, kidney cancer, liver cancer, lung cancer, mesothelioma, ovarian cancer, pancreatic cancer, skin cancer, thyroid cancer and uterine cancer.
